(19) United States
(12) Patent Application Publication
(10) Pub. No.: US 2004/0181820 A1
(43) Pub. Date: Sep. 16, 2004

(54) Nucleic acids for transforming fish cells and methods for their use

(76) Inventors: Marta Alonso, Eugene, OR (US); Pinwen P. Chiou, Storrs, CT (US); Jo-Ann C. Leong, Kaneohe, HI (US); Marc C Johnson, Ithaca, NY (US)

Correspondence Address: KLARQUIST SPARKMAN, LLP, 121 SW SALMON STREET, SUITE 1600, PORTLAND, OR, 97204, US

(21) Appl. No.: 10/469,884

(22) PCT Filed: Mar. 4, 2002
(86) PCT No.: PCT/US02/06738

Related U.S. Application Data
(60) Provisional application No. 60/273,584, filed on Mar. 5, 2001.

(51) Int. Cl. A01K 67/00; C07H 21/04; C07K 14/005

(52) U.S. Cl. 800/8; 435/69.3; 435/235.1; 435/320.1; 435/325; 530/350; 536/23.72

(57) ABSTRACT

A nucleic acid sequence, the rainbow trout interferon regulatory factor-1 (IRF1A) promoter, is disclosed. This promoter is capable of expressing a nucleic acid sequence operably linked to it in fish cells. IRF1A can be operably linked to antigenic sequences for fish or shellfish pathogens, thus inducing an immune response in a fish transformed with such a nucleic acid. Some of the vectors described utilize a nucleic acid containing an inducible promoter operably linked to a nucleic acid sequence encoding a polypeptide capable of inducing programmed cell death (PCD).

### FIELD

This invention relates to nucleic acid expression vectors for use in fish. Additionally, this invention relates to promoters and to expression vectors encoding antigenic epitopes for use in fish, including suicide vectors.

### BACKGROUND

The combined output of ocean fisheries and aquaculture must grow by ten to thirty percent by the year 2010 in order to feed a world population projected to increase by twelve percent from the approximately 6 billion people alive today. Ocean-based fisheries alone cannot support the expected increase in fish consumption, though the aquaculture industry can augment global fish production. Unfortunately, diseases infecting fish have a devastating economic impact on the aquaculture industry every year. For example, in the United States alone, estimated trout losses in 1997 totaled 7.84 million pounds, and 84% of those losses were due to diseases.

Fish populations are susceptible to a wide range of diseases caused by different pathogens. When fish are reared intensively in aquaculture systems, they are more vulnerable to these diseases. Strategies utilizing antibiotics and chemotherapeutic treatments can be used to control bacterial and parasitic diseases, but these products have been severely restricted by government-licensing agencies because they have undesirable effects such as accumulation in the flesh of the fish, increased appearance of antibiotic resistant fish pathogens, and contamination of the aquatic environment. Since many antibiotics are ineffective against viruses, antiviral treatments are needed for prevention of viral infectious diseases in aquaculture facilities around the world.

For viral infections, where there is no treatment available, the appearance of disease usually requires destruction of infected stock and decontamination of facilities. Consequently, antiviral treatments are desperately needed for prevention of infectious diseases in aquaculture worldwide. For the aquaculture industry, treatments for viral diseases are in a relative early phase of development. Many treatments have been tested under laboratory conditions, but most are not commercially viable because of their prohibitive cost of production, insufficient protection, or biosafety concerns.

Infectious hematopoietic necrosis virus (IHNV) is one common infectious agent of fish. An outbreak of IHNV can kill 80-100% of the fish at an aquaculture facility. Thus, safe and effective products and methods for protecting fish from IHNV (and other infectious agents) are needed.

### SUMMARY

Disclosed is a nucleic acid obtained from rainbow trout, an interferon regulatory factor-1 promoter (IRF1A; SEQ ID NO:1), and nucleic acids that have similar sequence identities, such as nucleic acids that are at least 70% identical to IRF1A (SEQ ID NO:1). Therefore, as used herein, the term “IRF1A promoter” encompasses the nucleic acid sequence provided by SEQ ID NO:1 and nucleic acids that are at least 70% identical to SEQ ID NO:1.

The IRF1A promoter may be operably linked to a heterologous nucleic acid, such as a nucleic acid encoding an antigenic epitope. One exemplary, non-limiting antigenic epitope is the infectious hematopoietic necrosis virus (IHNV) G protein or a conservative variant thereof. Additionally, the IRF1A promoter can be inserted into a vector, such as a plasmid or viral vector, and can drive expression of operably linked heterologous nucleic acid sequences contained on that vector.

The IRF1A promoter also can be inserted into a host cell, such as a fish cell, include vectors containing the IRF1A promoter and/or heterologous nucleic acids that are operably linked to the IRF1A promoter. In some embodiments, the host cell is the cell of a fish, thus producing a transgenic fish that includes the IRF1A promoter.

In particular embodiments, the IRF1A promoter is operably linked to a heterologous nucleic acid encoding an antigenic epitope and inserted into a vector. Such a vectors can be used to induce an immune response in fish against the antigenic epitope encoded by the nucleic acid sequence on the vector. In more particular embodiments, the vector includes another expression control sequence operably linked to a nucleic acid sequence encoding a polypeptide that induces programmed cell death (PCD), such as an IHNV M protein or a conservative variant thereof. In such embodiments, PCD can be induced by inducing expression of the nucleic acid sequence encoding the polypeptide that induces PCD. For example, and without limitation, an inducible promoter (such as a metallothionein promoter, heat shock promoter, carbonic anhydrase promoter, or a haptoglobin nucleic acid promoter) can be operably linked on the vector to the nucleic acid sequence encoding the polypeptide that induces PCD. Inducing the inducible promoter then drives expression of the polypeptide that induces PCD, thus leading to cell death.

### BRIEF DESCRIPTION OF THE DRAWINGS

FIG. 1 is the DNA sequence of the rainbow trout IRF1A promoter (SEQ ID NO:1) with certain functional elements identified.

FIG. 2 is a graph illustrating the effectiveness of certain nucleic acid compositions in conferring immunity to IHNV in fish.

FIG. 3 is a digital image of a PCR products run on an agarose gel. PCR analysis was used to detect the IHNV glycoprotein nucleic acid in fish transformed with a described vector.

FIG. 4 is a schematic illustration of two particular suicide vectors, pIRF1A-G-pMT-M and pCMV-G-pMT-M.

FIGS. 5a-f are digital images illustrating the effectiveness of certain suicide vectors in inducing apoptosis.

FIG. 6 is a schematic representation of the mechanism of action of a suicide vector.

FIG. 7 is a digital image of PCR products amplified from a suicide vector run on an agarose gel and stained with ethidium bromide. RT-PCR analysis was used to detect the persistence of the IHNV glycoprotein in cells transfected with 0.005 μg (lanes 1 and 2) or 0.01 μg (lanes 5, 6, 7 and 8) of the suicide vector. At 24 hours post-transfection, the cells were treated with 200 μM (lane 1), 100 μM (lane 5) or 150 μM (lane 7) of ZnCl₂. As a PCR positive control, PCR products from amplifying the plasmid originally used to inject the fish, pIRF-G-pMT-M, are shown in lane number 3. Lanes 4 and 9 are the PCR negative controls.

FIG. 8 is a digital image of PCR products amplified from a suicide vector run on an agarose gel and stained with ethidium bromide. PCR analysis was used to detect the IHNV glycoprotein nucleic acid in rainbow trout fry (0.3 g) injected with different concentrations of the suicide vector at 2 days after injection. The 1589 bp fragment was present in the two fish injected with 4 μg (lanes 1 and 2) and 1 μg (lanes 3 and 4). PCR positive results were obtained with one of the two fish (lanes 5 and 6) injected with 0.1 μg of the plasmid.

FIG. 9 is a digital image of PCR products amplified from a suicide vector run on an agarose gel and stained with ethidium bromide. PCR analysis was used to detect the persistence of the IHNV glycoprotein nucleic acid in rainbow trout fry (0.3 g) injected with 0.1 μg of the suicide DNA vaccine and treated 24 hour later with 100 μM of ZnCl₂. The 1589 bp fragment was present in the two fish vaccinated but non-treated with ZnCl₂ (lanes 1 and 2). The plasmid DNA was not detected in the muscle tissue of one of the two vaccinated and ZnCl₂ exposed fish (lanes 3 and 4). As a PCR positive control, the PCR results of the plasmid originally used to inject the fish, pIRF-G-pMT-M, are shown in lane number 5.

### BRIEF DESCRIPTION OF THE SEQUENCE LISTING

The nucleic acid sequences listed herein are shown using standard letter abbreviations for nucleotide bases. Only one strand of each nucleic acid sequence is shown, but the complementary strand is understood as included by any reference to the displayed strand.

SEQ ID NO: 1 shows the rainbow trout interferon regulatory factor-1 (IRF1A) promoter.

SEQ ID NO: 2 shows an IHNV-G specific PCR primer.

SEQ ID NO: 3 shows another IHNV-G specific PCR primer.

### DETAILED DESCRIPTION

A nucleic acid based antiviral treatment for protecting fish against viruses is disclosed. The treatment is effective against fish viruses generally, including (but not limited to) the infectious hematopoietic necrosis virus (IHNV), infectious pancreatic necrosis virus (IPNV), viral hemorrhagic septicemia virus (VHSV), infectious salmon anemia virus (ISA), and Hirama rhabdovirus (HIRRV).

Some antiviral treatments are already known, such as killed vaccines and subunit vaccines. For example, Anderson et al. developed an effective DNA-based treatment against IHNV. Anderson, et al., Mol. Mar. Biol. Biotechnol. 5(2):114-22 (1996). That treatment consisted of an expression vector including the nucleic acid for the IHNV surface glycoprotein (IHNV G) under transcriptional control of the cytomegalovirus immediate early promoter (CMVIEP). However, CMVIEP is a strong promoter that primes transcription in a wide variety of eukaryotic cell types. CMVIEP, derived from a human pathogenic virus, may be publicly perceived as unsafe for use in treating fish destined for human consumption.

In contrast, some embodiments disclosed herein utilize a novel promoter, the rainbow trout interferon regulatory factor-1 (IRF1A) promoter, operably linked to a nucleic acid encoding a polypeptide, such as a viral antigenic epitope. In one embodiment, IRF1A is operably linked to the infectious hematopoietic necrosis virus surface glycoprotein (IHNV G), or a conservative variant thereof.

Other embodiments utilize an expression control sequence operably linked to a nucleic acid sequencing encoding a polypeptide, and a second expression control sequence operably linked to a nucleic acid encoding a protein capable of inducing cell death, such as the infectious hematopoietic necrosis virus matrix protein (IHNV M), or a conservative variant thereof. In several of these embodiments, the second expression control sequence is an inducible promoter, such as the metallothionein (MT) promoter, the fish heat shock protein 70 (HSP70) highly inducible promoter, the carbonic anhydrase (CA) promoter, or the haptoglobin nucleic acid promoter.

Explanations of Terms

The following explanations of terms are provided to better illustrate the present invention. Definitions of common terms also can be found in Rieger et al., Glossary of Genetics: Classical and Molecular, 5th edition, Springer-Verlag: New York, 1991; Lewin, Genes VII, Oxford University Press: New York, 1999; Dictionary of Bioscience, Mcgraw-Hill: New York 1997; Kendrew et al (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The standard one- and three-letter nomenclature for amino acid residues is used.

The singular forms “a,” “an,” and “the” refer to one or more than one, unless the context clearly dictates otherwise. For example, the term “comprising a nucleic acid” includes single or plural nucleic acids and is considered equivalent to the phrase “comprising at least one nucleic acid.”

The term “or” refers to a single element of stated alternative elements or a combination of two or more elements. For example, the phrase “a first nucleic acid or a second nucleic acid” refers to the first nucleic acid, the second nucleic acid, or both the first and second nucleic acids.

As used herein, “comprises” means “includes.” Thus, “comprising A and B” means “including A and B,” without excluding additional elements.

Conservative amino-acid substitution. Conservative amino acid substitutions in a polypeptide, such as IHNV G or IHNV M, include those listed in Table 1 below.

TABLE 1

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Non-conservative substitutions are those that disrupt the secondary, tertiary, or quaternary conformation of a polypeptide. Such non-conservative substitutions can result from changes in: (a) the structure of the polypeptide backbone in the area of the substitution; (b) the charge or hydrophobicity of the polypeptide; or (c) the bulk of an amino acid side chain. Substitutions generally expected to produce the greatest changes-in polypeptide properties are those in which: (a) a hydrophilic residue is substituted for (or by) a hydrophobic residue; (b) a proline is substituted for (or by) any other residue; or (c) a residue having a bulky side chain, e.g., phenylalanine, is substituted for (or by) one not having a side chain, e.g., glycine. In particular embodiments, a residue having an electropositive side chain, e.g., lysyl, arginyl, or histadyl, is not substituted for (or by) an electronegative residue, e.g., glutamyl or aspartyl.

Amphotrophic virus. A virus that can replicate both in cells of its native host and also in cells of other species.

Amplification: Regarding a nucleic acid molecule (e.g., a DNA or RNA molecule), amplification refers to use of a technique that increases the number of copies of a nucleic acid molecule in a specimen. An example of amplification is the polymerase chain reaction (PCR), in which a biological sample collected from a subject is contacted with a pair of oligonucleotide primers under conditions that allow for the hybridization of the primers to nucleic acid template in the sample. The primers are extended under suitable conditions, dissociated from the template, and then re-annealed, extended, and dissociated to amplify the number of copies of the nucleic acid. The product of amplification can be characterized by electrophoresis, restriction endonuclease cleavage patterns, oligonucleotide hybridization or ligation, and/or nucleic acid sequencing using standard techniques. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in WO 90/01069; ligase chain reaction amplification, as disclosed in EP-A-320 308; gap filling ligase chain reaction amplification, as disclosed in U.S. Pat. No. 5,427,930; and NASBA™ RNA transcription-free amplification, as disclosed in U.S. Pat. No. 6,025,134.

Analog or homolog. An analog is a molecule that differs in chemical structure from a parent compound. A homolog differs by an increment in the chemical structure (such as a difference in the length of a nucleic acid or amino acid chain), a molecular fragment, a structure that differs by one or more functional groups, or a change in ionization.

Animal. A living, multi-cellular, vertebrate organism, including, for example, mammals, birds, reptiles, and fish. The term “fish” includes both Chondrichthyes and Osteichthyes, though in particular embodiments, the fish is a member of Osteichthyes. The term “mammal” includes both human and non-human mammals. Similarly, the term “subject” includes both human and veterinary subjects.

Antigen. A compound, composition, or substance that can stimulate the production of antibodies or a T-cell response in an animal, including compositions that are injected or absorbed into an animal. An antigen reacts with the products of specific humoral or cellular immunity, including those induced by heterologous immunogens. The term “antigen” includes all related antigenic epitopes.

Delivery of compositions. For administration to animals, purified active compositions can be administered alone or combined with an acceptable carrier. Preparations can contain one type of therapeutic molecule, or can be composed of a combination of several types of therapeutic molecules. The nature of the carrier will depend on the particular mode of administration being utilized. For instance, parenteral formulations usually comprise injectable fluids that include physiologically acceptable fluids such as water, physiological saline, balanced salt solutions, aqueous dextrose, glycerol or the like as a vehicle. For solid compositions (e.g., powder, pill, tablet, or capsule forms), which can be added to an aquaculture environment, conventional non-toxic solid carriers can include, for example, mannitol, lactose, starch, or magnesium stearate. In addition to biologically-neutral carriers, compositions to be administered to fish can contain minor amounts of non-toxic auxiliary substances, such as wetting or emulsifying agents, preservatives, and pH buffering agents and the like, for example sodium acetate or sorbitan monolaurate.

It is also contemplated that the nucleic acids could be delivered to cells subsequently expressed by the host cell, for example through the use viral vectors, plasmid vectors, or liposomes administered to fish.

Compositions of the present invention can be administered by any means that achieve their intended purpose. Amounts and regimens for the administration of the nucleic acids, or an active fragment thereof, can be readily determined.

For use in treating viral infections, compositions are administered in an amount effective to inhibit viral infection or progression of an existing infection, or administered in an amount effective to inhibit or alleviate a corresponding disease. In one embodiment, infection is completely prevented.

Typical amounts initially administered would be those amounts adequate to achieve tissue concentrations at the site of action which have been found to achieve the desired effect in vitro. The compositions can be administered to a host in vivo, for example through systemic administration, such as intravenous, intramuscular, or intraperitoneal administration. The compositions also can be administered intralesionally, through scarification of the skin, intrabuccal administration, cutaneous particle bombardment, or by immersion in water containing a nucleic acid composition described herein (for uptake by the fish). Additionally, the nucleic acid compositions can be administered by encapsulation with a nanoparticle matrix composed of a nucleic acid in methacrylic acid polymer, and an attenuated bacteria (such as Yersinia ruckeri, Edwardsiella ictaluri, Aeromonas salmonicida, or Vibrio anguillarum) carrying the nucleic acid for delivery by immersion administration (see, e.g., U.S. Pat. No. 5,877,159, herein incorporated by reference).

Effective doses for using compositions can vary depending on the nature and severity of the condition to be treated, the age and physiological condition of the fish, mode of administration, and other relevant factors. Thus, the final determination of the appropriate treatment regimen can be made by someone at the site of the fish, such as an operator or employee of an aquaculture facility. Typically, the dose range will be from about 1 μg/kg body weight to about 100 mg/kg body weight, such as about 10 μg/kg body weight to about 900 μg/kg body weight, or from about 50 μg/kg body weight to about 500 μg/kg body weight, or from about 50 μg/kg body weight to about 150 μg/kg body weight, such as about 100 μg/kg body weight. Nanogram quantities of transforming DNA have been shown to be capable of inducing an immune response in fish (see, e.g., Corbeil, S., et al., Vaccine 18(25):2817-24 (2000), herein incorporated by reference).

The dosing schedule can vary from a single dosage to multiple dosages given several times a day, once a day, once every few days, once a week, once a month, annually, biannually, biennially, or any other appropriate periodicity. The dosage schedule can depend on a number of factors, such as the species' or subject's sensitivity to the composition, the type and severity of infection, route of administration, and the volume of the container that contains the fish. In the case of a more aggressive disease, compositions can be administered by alternate routes, including intramuscularly and by environmental uptake. Continuous administration also can be appropriate in some circumstances.

cDNA (complementary DNA). A piece of DNA lacking internal, non-coding segments (introns) and regulatory sequences that determine transcription. cDNA can be synthesized in a laboratory by reverse transcription from messenger RNA extracted from cells.

Complementarity. A nucleic acid that performs a similar function to the sequence to which it is complementary. The complementary sequence does not have to confer replication competence in the same cell type to be complementary, but merely confer replication competence in some cell type.

Epitope. A site on an antigen at which an antibody can bind, the molecular arrangement of the site determining the combining antibody. A portion of an antigen molecule that determines its capacity to combine with the specific combining site of its corresponding antibody in an antigen-antibody interaction.

Hybridization. Nucleotide molecules and sequences which are derived from the disclosed nucleotide molecules as described above also can be defined as nucleotide sequences that hybridize to the nucleotide sequences disclosed, or fragments thereof. In particular embodiments, the hybridization is done under stringent conditions, as described further below.

Hybridization conditions resulting in particular degrees of stringency will vary depending upon the nature of the hybridization method of choice and the composition and length of the hybridizing nucleic acid used. Generally, the temperature of hybridization and the ionic strength (especially the Na⁺ concentration) of the hybridization buffer will determine the stringency of hybridization. Calculations regarding hybridization conditions required for attaining particular degrees of stringency are discussed by Sambrook et al. (2001), chapters 9 and 11, herein incorporated by reference. By way of illustration only, a hybridization experiment can be performed by hybridization of a DNA molecule (for example, a variation of the IRF1A of SEQ ID NO:1) to a target DNA molecule (for example, a putative promoter sequence similar to IRF1A) which has been electrophoresed in an agarose gel and transferred to a nitrocellulose membrane by Southern blotting, a technique well known in the art and described in Sambrook et al., 2001. Hybridization with a target probe labeled with [³²P]-dCTP is generally carried out in a solution of high ionic strength such as 6×SSC at a temperature that is 20-25° C. below the melting temperature, Tₘ, described below. For such Southern hybridization experiments, where the target DNA molecule on the Southern blot contains 10 ng of DNA or more, hybridization is typically carried out for 6-8 hours using 1-2 ng/ml radiolabeled probe (of specific activity equal to 10⁹ CPM/μg or greater). Following hybridization, the nitrocellulose filter is washed to remove background hybridization. The washing conditions should be as stringent as possible to remove background hybridization yet retain a specific hybridization signal. The term Tₘ represents the temperature above which, under the prevailing ionic conditions, the radiolabeled probe molecule will not hybridize to its target DNA molecule. The Tₘ of such a hybrid molecule can be estimated from the following equation:

Tₘ=81.5C−16.6(log₁₀[Na⁺])+0.41(% G+C)−0.63(% formamide)−(600/l) Where l=the length of the hybrid in base pairs.

This equation is valid for concentrations of Na⁺ in the range of 0.01 M to 0.4 M, and it is less accurate for calculations of Tₘ in solutions of higher [Na⁺]. The equation is also primarily valid for DNAs whose G+C content is in the range of 30% to 75%, and it applies to hybrids greater than 100 nucleotides in length (the behavior of oligonucleotide probes is described in detail in Ch. 9 of Sambrook et al., 2001).

The Tₘ of double-stranded DNA decreases by 1-1.5° C. with every 1% decrease in homology. Therefore, for this given example, washing the filter in 0.3×SSC at 59.4-64.4° C. will produce a stringency of hybridization equivalent to 90%; that is, DNA molecules with more than 10% sequence variation relative to the target DLC-1 cDNA will not hybridize. Alternatively, washing the hybridized filter in 0.3×SSC at a temperature of 65.4-68.4° C. will yield a hybridization stringency of 94%; that is, DNA molecules with more than 6% sequence variation relative to the target DLC-1 cDNA molecule will not hybridize.

The above example is given entirely by way of theoretical illustration. Other hybridization techniques can be utilized, and variations in experimental conditions may necessitate alternative calculations for stringency.

The degeneracy of the genetic code further widens the scope of the present invention as it enables major variations in the nucleotide sequence of a DNA molecule while maintaining the amino acid sequence of the encoded protein. Thus, the nucleotide sequence encoding IHNV G could be changed without affecting the amino acid composition of the encoded polypeptide or the characteristics of the polypeptide. The genetic code and variations in nucleotide codons for particular amino acids is known. Based upon the degeneracy of the genetic code, variant DNA molecules can be derived using standard techniques, such as DNA mutagenesis techniques or synthesis of DNA sequences. DNA sequences that do not hybridize under stringent conditions to the DNA sequences disclosed herein, by virtue of sequence variation based on the degeneracy of the genetic code, also are comprehended by this invention.

“Stringent conditions” encompass conditions under which hybridization will only occur if there is less than 25% mismatch between the hybridization nucleic acid sequence and the target nucleic acid sequence. “Stringent conditions” can be broken down into particular levels of stringency for more precise measurement. Thus, as used herein, “moderate stringency” conditions are those under which nucleic acid molecules with more than 25% sequence variation (also termed “mismatch”) will not hybridize; conditions of “medium stringency” are those under which nucleic acid molecules with more than 15% mismatch will not hybridize, and conditions of “high stringency” are those under which nucleic acid sequences with more than 10% mismatch will not hybridize. Conditions of “very high stringency” are those under which nucleic acid sequences with more than 6% mismatch will not hybridize.

GenBank. GenBank is the National Institutes of Health (NIH) genetic sequence database, an annotated collection of all publicly available DNA sequences (Nucleic Acids Res., 28(1):15-18 (2000)). References to GenBank contained herein refer to GenBank Flat File Release 122.0, dated 15 Feb. 2001.

Genetic fragment. Any nucleic acid derived from a larger nucleic acid.

Heterologous. Originating from a different organism or distinct tissue culture, such as from a different species or cell line.

Homologs. Two nucleotide sequences that share a common ancestral sequence and diverged when a species carrying that ancestral sequence split into two species.

IHNV G and IHVN M. The infectious hematopoietic necrosis virus glycoprotein (IHNV G) is a surface polypeptide, or virion protein, of IHNV that interacts with a host receptor to mediate virus entry into a cell. IHNV G contains carbohydrate moieties and is encoded by the fourth nucleic acid, G, in the IHNV genome, whose order is 3′-N-P-M-G-NV-L-5′, where N is the nucleocapsid protein nucleic acid, P is the phosphorprotein nucleic acid, M is the matrix protein nucleic acid, G is the glycoprotein nucleic acid, NV is the non-virion protein nucleic acid, and L is the RNA polymerase nucleic acid.

IHNV is responsible for the induction of neutralizing antibodies and is capable of inducing an immune response in animal cells against IHNV infection (see, e.g., Koener, J. F., et al., J. Virol., 61(5):1342-49 (1987)). The amino acid sequence for IHNV G is shown as GenBank Accession No. U50401, herein incorporated by reference. The hematopoietic necrosis virus matrix protein (IHNV M) has been described as a programmed cell death inducer (see Chiou P. P., et al., J Virol. 74(16):7619-27 (2000)). The amino acid sequence for IHNV M is shown as GenBank Accession No. X73872, herein incorporated by reference.

IHNV G and IHNV M polypeptides can be isolated from cells or synthesized using standard techniques Alternatively, IHVN G and IHNV M polypeptides can be produced by standard genetic engineering techniques (e.g., by the expression of a nucleic acid sequence encoding IHNV G and/or IHNV M in an appropriate host cell). The polypeptide can be expressed as a fusion polypeptide if its activity is not significantly diminished by the fusion partner. For guidance regarding expression of polypeptides in various host cells (see, e.g., Molecular Cloning: A Laboratory Manual, 3rd ed., vol. 1-3, ed. Sambrook, et al. (Cold Spring Harbor Laboratory Press, Cold Spring Harbor, N.Y., 2001) and Current Protocols in Molecular Biology, ed. Ausubel et al., with periodic updates (Greene Publishing and Wiley-Interscience, New York, 1987)).

Isolated. An “isolated” biological component (such as a nucleic acid, polypeptide, protein, or organelle) has been substantially separated, produced apart from, or purified away from other biological components (e.g., other chromosomal and extrachromosomal DNA and RNA, and polypeptides) found in the cell of the organism in which the component naturally occurs. Nucleic acids, polypeptides, and proteins that have been “isolated” thus include nucleic acids and polypeptides purified by standard purification methods. The term also embraces nucleic acids, polypeptides, and proteins that are chemically synthesized or prepared by recombinant expression in a host cell.

Nucleic acid. A deoxyribonucleotide or ribonucleotide polymer in either single or double stranded form. Unless otherwise limited, this term encompasses known analogues of natural nucleotides that hybridize to nucleic acids in a manner similar to naturally occurring nucleotides. An “oligonucleotide” (or “oligo”) is a linear nucleic acid of up to about 250 nucleotide bases in length. For example, a nucleic acid (such as DNA or RNA) that is at least 5 nucleotides long, such as at least 15, 50, 100, or even more than 200 nucleotides long.

Operably linked. A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous. Where necessary to join two protein coding regions, the operably linked sequences are in the same reading frame.

Expression control sequence. A nucleic acid sequence that affects, modifies, or influences expression of a second nucleic acid sequence. Promoters, operators, repressors, and enhancers are examples of expression control sequences.

In some embodiments, the expression control sequence is a promoter, such as an IRF1A promoter.

ORF (open reading frame). A series of nucleotide triplets (codons) coding for amino acids without any termination codons. These sequences are usually translatable into a peptide.

Ortholog. Two nucleotide sequences are orthologs of each other if they share a common ancestral sequence and diverged when a species carrying that ancestral sequence split into two species. Orthologous sequences are also homologous sequences.

Parenteral. Administered outside of the intestine, e.g., not via the alimentary tract. Generally, parenteral formulations are those that will be administered through any possible mode except ingestion. This term especially refers to injections, whether administered intravenously, intrathecally, intramuscularly, intraperitoneally, or subcutaneously, and various surface applications including intranasal, intradermal, and topical application, for instance.

Polypeptide. Any chain of amino acids, regardless of length or post-translational modification (e.g., glycosylation or phosphorylation).

Polypeptide sequence somology. Preferably, a polypeptide according to the present invention is at least about 70% homologous to a corresponding sequence (such as SEQ ID NO:1) or a native polypeptide (such as IHNV G), such as at least about 80% homologous, and even at least about 95% homologous. Such homology is considered to be “substantial homology.”

Polypeptide homology is typically analyzed using sequence analysis software, such as the programs available from the Genetics Computer Group (Madison, Wis.).

Portion of a nucleic acid sequence. At least 10, 20, 30, 40, 50, 60, 70, 80, or more contiguous nucleotides of the relevant sequence. In some embodiments, a portion consisting of 50 or more nucleotides is used. For example, a portion of the IRF1A promoter sequence can be described as comprising at least 50 nucleotides in a particular region of the entire nucleic acid sequence, such as the sequence encompassing the NF-kβ element and the TATA sequence (i.e., from −103 to −78 in SEQ ID NO:1) or the sequence encompassing the two imperfect inverted repeats (i.e., from −210 to −180 in SEQ ID NO:1), up to a length that includes one entire contiguous fragment of the sequence between the Sp1 motif and the TATA consensus sequence (i.e., from −1 186 to −78 in SEQ ID NO:1). As another example, in the IHNV G polypeptide, an immunodominant epitope is located in the from about amino acid 269 to about amino acid 453. When synthetic IHNV G peptides have been examined, a sequence from about amino acid 306 to about amino acid 326 induced neutralizing antibody in both mice and fish. Additionally, monoclonal antibodies have been used to select for escape mutants of IHNV and two antigenic sites were identified, one at about amino acid 230-231 and another at about amino acid 272-276 (see Xu, L., et al., J. Virol. 65(3):1611-1615 (1991); Mourich, D. V., and Leong, J. C., Proceedings of the Second International Symposium on Viruses of Lower Vertebrates, 29-31 Jul. 1991, Corvallis, OR, pp. 93-100 (1991); Huang, C., et al., J. Gen. Virol. 77(12):3033-40 (1996), all three of which are herein incorporated by reference).

Promoter. A promoter is one type of expression control sequence composed from an array of nucleic acid sequences that directs transcription of a nucleic acid. A promoter includes necessary nucleic acid sequences near the start site of transcription, such as, in the case of an IRF1A promoter, a TATA element. A promoter also can include distal enhancer or repressor elements that can be located as much as several thousand base pairs from the start site of transcription.

An inducible promoter directs transcription of a nucleic acid operably linked to it only under certain environmental conditions, such as in the presence of metal ions or above a certain temperature.

Protein Purification. The polypeptides of the present invention can be purified by any of the means known in the art (see, e.g., Guide to Protein Purification: Methods Enzymologyl, ed. Deutscher, Academic Press, San Diego, 1997; and Scopes, Protein Purification: Principles and Practice, 3^{rd} ed., Springer Verlag, N.Y., 1994).

Purified. The term purified does not require absolute purity; rather, it is intended as a relative term. Thus, for example, a purified nucleic acid is one in which the nucleic acid is more enriched than the nucleic acid is in its natural environment within a cell. Preferably, a preparation is purified such that the nucleic acid represents at least 50% of the total nucleic acid content of the preparation.

Recombinant. A recombinant nucleic acid is one that has a sequence that is not naturally occurring or has a sequence that is made by an artificial combination of two otherwise separated segments of sequence. This artificial combination can be accomplished by chemical synthesis or artificial manipulation of isolated segments of nucleic acids, e.g., by genetic engineering techniques. Similarly, a recombinant protein is one encoded for by a recombinant nucleic acid molecule. The term recombinant includes nucleic acids that have been altered solely by deletion of a portion of the nucleic acid.

Resistance to infection. Animals resistant to infection will demonstrate decreased symptoms of infection compared to non-resistant animals. Evidence of resistance to infection can appear as, for example, lower rates of mortality; increased life-spans measured after exposure to the infective agent; fewer or less intense physiological symptoms, such as fewer lesions; or decreased cellular or tissue concentrations of the infective agent. Additionally, a heightened immune response often suggests a resistance to infection.

Sequence identity. The similarity between two nucleic acid sequences, or two amino acid sequences, is expressed in terms of the similarity between the sequences, otherwise referred to as sequence identity. Sequence identity is frequently measured in terms of percentage identity (or similarity or homlogy); the higher the percentage, the more similar are the two sequences.

Methods of alignment of sequences for comparison are well-known in the art. Various programs and alignment algorithms are described in: Smith and Waterman, Adv. Appl. Math. 2:482, 1981; Needleman and Wunsch, J. Mol. Bio. 48:443, 1970; Pearson and Lipman, Methods in Molec. Biology 24: 307-331, 1988; Higgins and Sharp, Gene 73:237-244, 1988; Higgins and Sharp, CABIOS 5:151-153, 1989; Corpet et al., Nucleic Acids Research 16:10881-90, 1988; Huang et al., Computer Applications in BioSciences 8:155-65,1992; and Pearson et al., Methods in Molecular Biology 24:307-31,1994. Altschul et al. (1994) presents a detailed consideration of sequence alignment methods and homology calculations.

The NCBI Basic Local Alignment Search Tool (BLAST) (Altschul et al., J. Mol. Biol. 215:403-410, 1990) is available from several sources, including the National Center for Biological Information (NCBI, Bethesda, Md.) and on the Internet, for use in connection with the sequence analysis programs blastp, blastn, blastx, tblastn and tblastx. It can be accessed via the NCBI website.

Homologs of the nucleic acids and polypeptides described herein are typically characterized by possession of at least 70% sequence identity counted over the full length alignment with a disclosed sequence using the NCBI Blast 2.0, gapped blastp set to default parameters. Such homologous nucleic acids or peptides will possess at least 70%, at least 80%, or even at least 90% or 95% sequence identity determined by this method. When less than the entire sequence is being compared for sequence identity, homologs will possess at least 70%, such as at least 85%, or even at least 90% or 95% sequence identity over short windows of 10-20 amino acids. Methods for determining sequence identity over such short windows are described at the NCBI website. These sequence identity ranges are provided for guidance only; it is entirely possible that strongly significant homologs or other variants could be obtained that fall outside of the ranges provided.

The present invention provides not only the peptide homologs that are described above, but also nucleic acid molecules that encode such homologs. One indication that two nucleic acid sequences are substantially identical is that the polypeptide which the first nucleic acid encodes is immunologically cross reactive with the polypeptide encoded by the second nucleic acid. Another indication that two nucleic acid sequences are substantially identical is that the two molecules hybridize to each other under stringent conditions. Stringent conditions, as described above, are sequence dependent and are different under different environmental parameters.

Nucleic acid sequences that do not show a high degree of identity can nevertheless encode similar amino acid sequences, due to the degeneracy of the genetic code. It is understood that changes in nucleic acid sequence can be made using this degeneracy to produce multiple nucleic acid sequence that all encode substantially the same polypeptide.

Substantially similar. When optimally aligned (with appropriate nucleotide insertions or deletions) with the other nucleic acid (or its complementary strand), there is nucleotide sequence identity in at least about 50%, 60%, 70%, 80% or 90 to 95% of the nucleotide bases.

Therapeutic agent. Includes treating agents, prophylactic agents, and replacement agents made from nucleic acid compositions described herein.

Therapeutically effective amount or effective amount. A quantity sufficient to achieve a desired effect in situ, in vitro, in vivo, or within a subject being treated. For instance, the effective amount can be the amount necessary to inhibit viral proliferation or to measurably neutralize progression of disease. In general, this amount will be sufficient to measurably inhibit virus (e.g., IHNV) replication or infectivity.

An effective amount can be administered in a single dose, or in several doses, for example daily, during a course of treatment. However, the effective amount can depend on the composition applied or administered, the subject being treated, the severity and type of the affliction, and the manner of administration.

The compositions disclosed in the present invention have application in various settings, such as aquaculture, environmental containment, or veterinary settings. Therefore, the general term “subject being treated” is understood to include all fish that are or can be infected with a virus or other disease-causing microorganism that is susceptible to neutralization by the compositions described herein.

Transduced, transformed, and transfected. A virus or vector “transduces” a cell when it transfers nucleic acid into the cell. A cell is “transformed” by a nucleic acid transduced into the cell when the DNA becomes stably replicated by the cell, either by incorporation of the nucleic acid into the cellular genome, or by episomal replication. Transfection is the uptake by eukaryotic cells of a nucleic acid from the local environment and can be considered the eukaryotic counterpart to bacterial transformation.

As used herein, the term transformation encompasses all techniques by which a nucleic acid molecule might be introduced into a cell.

Transgene. An exogenous nucleic acid supplied by a vector.

Transgenic. Of, pertaining to, or containing a nucleic acid, ORF, or other nucleic acid native to another species, microorganism, or virus. The term “transgenic” includes transient and permanent transformation, where the nucleic acid integrates into chromosomal DNA, including the germ line, or is maintained extrachromosomally. For example, fish transformed with a plasmid encoding an IHNV G or IHNV M polypeptide maintained extrachromosomally are understood to be transgenic.

Variants of Amino Acid and Nucleic Acid Sequences. The production of proteins disclosed herein (e.g., IHNV G and IHNV M polypeptides) can be accomplished in a variety of ways. DNA sequences which encode for the protein, or a fragment of the protein, can be engineered such that they allow the protein to be expressed in eukaryotic cells, bacteria, insects, and/or plants. In order to accomplish this expression, the DNA sequence can be altered and operably linked to other regulatory sequences. The final product, which contains the regulatory sequences and the nucleic acid, is referred to as a vector. This vector can then be introduced into the eukaryotic cells, bacteria, insect, and/or plant. Once inside the cell, the vector allows the protein to be produced.

The DNA can be altered in numerous ways without affecting the biological activity of the encoded protein. For example, PCR can be used to produce variations in the DNA sequence which encodes IHNV G or IHNV M. Such variants can be variants that are optimized for codon preference in a host cell that is to be used to express the protein, or other sequence changes that facilitate expression.

Two types of cDNA sequence variant can be produced. In the first type, the variation in the cDNA sequence is not manifested as a change in the amino acid sequence of the encoded polypeptide. These silent variations are simply a reflection of the degeneracy of the genetic code. In the second type, the cDNA sequence variation does result in a change in the amino acid sequence of the encoded protein. In such cases, the variant cDNA sequence produces a variant polypeptide sequence. In order to preserve the functional and immunologic identity of the encoded polypeptide, it is preferred that any such amino acid substitutions are conservative.

Variations in the cDNA sequence that result in amino acid changes, whether conservative or not, should be minimized in order to preserve the functional and immunologic identity of the encoded protein. Variant amino acid sequences can, for example, be 70, 80%, 90%, or even 95% identical to the native amino acid sequence.

Vector. A nucleic acid molecule as introduced into a host cell, thereby producing a transformed host cell. A vector can include nucleic acid sequences that permit it to replicate in the host cell, such as an origin of replication. A vector can also include one or more therapeutic genes and/or selectable marker genes and other genetic elements known in the art. A vector can transduce, transform or transfect a cell, thereby causing the cell to express nucleic acids and/or proteins other than those native to the cell. A vector optionally includes materials to aid in achieving entry of the nucleic acid into the cell, such as a viral particle, liposome, protein coating, or the like. Plasmids are often used as vectors to transform fish cells.

IRF1A

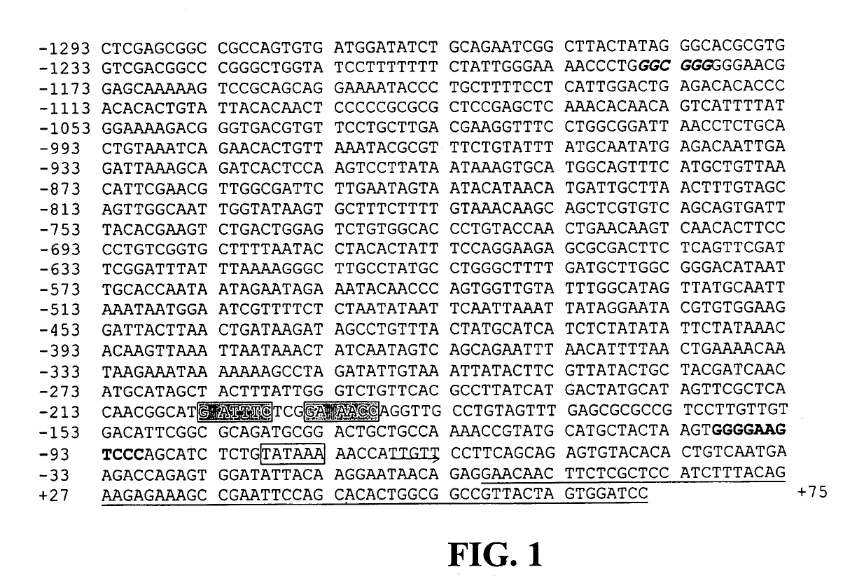

The nucleic acid sequence for the rainbow trout interferon regulatory factor-1 (IRF1A) promoter is provided herein. In one embodiment, an IRF1A promoter has a sequence as set forth as SEQ ID NO:1 and further illustrated in FIG. 1.

In one embodiment, when operatively linked to a heterologous nucleic acid sequence, the IRF1A promoter drives transcription of the heterologous nucleic acid sequence. In one embodiment, an IRF1A promoter has a sequence as set forth as SEQ ID NO:1. In others embodiment, an IRF1A promoter includes variants, such as nucleic acids sequences that differ from SEQ ID NO:1 by at most 5, at most 10, at most 20, at most 50, at most 75, or at most 100 nucleotides. These variants, when operably linked to a heterologous nucleic acid sequence, drive transcription of the heterologous nucleic acid sequence.

FIG. 1 presents the sequence and putative functional elements of IRF1A promoter. The arrow indicates the transcriptional site for the IRF1A mRNA. The TATA consensus sequence is boxed. An NF-kβ element, a potential transcription factor binding site, is shown in bold. An Sp1 motif is shown in italic, and two imperfect inverted repeats are marked in a gray box. Thus, in one embodiment, an IRF1A promoter includes the NF-kβ element and the TATA sequence (i.e., from −103 to −78 of SEQ ID NO:1), or a variant thereof, that when operably linked to a heterolgous nucleic acid sequence allows transcription of the heterologous nucleic acid sequence. In another embodiment, an IRF1A promoter includes the sequence encompassing the two imperfect inverted repeats (i.e., from −210 to −180 in FIG. 1) of the IRF1A promoter, or a variant thereof. In another embodiment, an IRF1A promoter includes a fragment of IRF1A promoter that includes the nucleic acid between the Sp1 motif and the TATA consensus sequence (i.e., from −1186 to −78 in FIG. 1), or a variant thereof.

An IRF1A sequence can be isolated by any method known to one of skill in the art. A specific, non-limiting example of a method of isolating the IRF1A sequence is described in Example 2. An IRF1A sequence, such as a nucleic acid sequence isolated from other fish species, chemically synthesized, or recombinantly expressed, also can be used, including nucleic acids that are 70%, 80%, 90%, or even 95% identical to SEQ ID NO:1. Thus in one specific, non-limiting example, an IRF1A promoter includes a sequence 70% identical to SEQ ID NO:1, that drives transcription of a heterologous nucleic acid sequence when operably linked to the heterologous nucleic acid sequence.

As noted above, IRF1A can be operably linked to a heterologous nucleic acid, such as a nucleic acid sequence encoding a polypeptide. In some embodiments, IRF1A is linked to a heterologous nucleic acid encoding a polypeptide, or functional protein, useful in enhancing the longevity, disease resistance, or performance of aquacultured animals. Aquacultural animals include: salmonids, such as rainbow trout (Oncorhynchus mykiss), coho salmon (O. kisutch), chinook salmon (O. tshawytcha), amago salmon (O. rhodurus), chum salmon (O. keta Walbaum), sockeye salmon (O. nerka), Atlantic salmon (Salmo salar), arctic char (Salvelinus alpinus), brown trout (Salmo trutta fario), cutthroat trout (Salmo clarkii), and brook trout (Salvelinus fontinalis); catfish (Ictalurus punctatus); tilapia (Oreochromis niloticusand and Oreochromis mozambicus); sea bream (Archosargus rhomboidalis), seabass (Dicentrarchus labrax); flounder (Paralichthys dentatus); and sturgeon (Scaphirhynchus albus).

IRF1A also can be operably linked to other heterologous nucleic acids, such as reporter genes or sequences encoding particular antigens, growth factors, hormones, enzymes, lytic-peptides, antibiotic cytokines, beta-ketolases, and antimicrobial peptides (e.g., cecropin, melittins, magainin, and defensins).

In certain embodiments, IRF1A is operably linked to antigenic sequences for fish or shellfish pathogens, such as sequences encoding all or antigenic components of the VP-2 nucleic acid of IPNV (GenBank Accession No. S58170, herein incorporated by reference); the glycoprotein genes of the different fish rhabdoviruses, including spring viremia of carp virus, hirame rhabdovirus, infectious salmon anemia virus, channel catfish virus, and viral hemorrhagic septicemia virus (VHSV) (the VHSV G nucleic acid is shown by GenBank Accession No. X66134, herein incorporated by reference). In other embodiments, IRF1A is operably linked to sequences encoding polypeptides for enhancing fish performance, such as the trout growth hormone (GenBank Accession No. M22731, herein incorporated by reference). In still other embodiments, IRF1A is operably linked to sequences encoding antimicrobial polypeptides, such as misgurin and pleurocin (see Jia, X., et al., Appl Environ Microbiol 66(5): 1928-32 (2000), herein incorporated by reference). In specific embodiments, IRF1A is operably linked to a nucleic acid sequence encoding the IHNV G protein, an antigenic fragment thereof, or a conservative variant thereof. Other embodiments utilize heterologous nucleic acids encoding an IHNV M polypeptide, or conservative variants thereof. Such compositions are described in further detail below.

A nucleic acid composition containing IRF1A can contain other elements, such as additional expression control elements, structural sequences, origins of replication, or multiple coding sequences.

In some embodiments, the nucleic acid composition containing IRF1A is utilized as an expression vector, such as a plasmid or viral vector, a phagemid, or a cosmid. Vectors include, but are not limited to, cloning vectors, such as those described in U.S. Pat. No. 5,998,697. Vectors also include viral vectors such as retroviral or adenoviral vectors.

The nucleic acid compositions described herein, such as compositions containing the IRF1A promoter, or the vectors described below, can be utilized in vitro, in vivo, or in situ. For example, IRF1A could be used to drive expression of a nucleic acid sequence of interest for in vitro production and manipulation, to study its effect on cell physiology or activity in vivo, or for tissue-specific expression analysis in situ. Particular uses of IRF1A are illustrated in the Examples below.

IRF1A activity has been shown in fish cells. For example, IRF1A activity has been demonstrated in salmonid salmonid cells, such as Chinook salmon cells (e.g., the CHSE-214 cell line; see Example 1 below) and rainbow trout cells (see Example 3 below), as well as other types of fish cells, such as carp cells (for example, EPC cells; see Example 6 below).

Antigenic Vectors

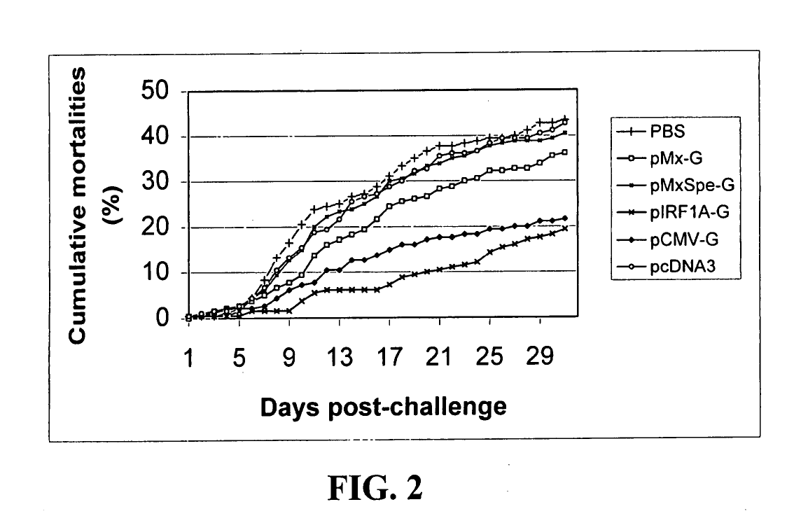

IRF1A can be operably linked to a heterologous coding sequence encoding an antigenic epitope, such as the IHNV glycoprotein (IHNV G) polypeptide, the IPNV VP-2 polypeptide, or the VHSV G polypeptide. Each of these polypeptides, when expressed in a fish, is capable of inducing an immune response to the corresponding pathogen. As just one example, IHNV G is capable of inducing an immune response in fish, such as rainbow trout, and conferring resistance to IHNV infection (see, e.g., Koener, J. F., et. al., J. Virol. 61(5):1342-49 (1987)). As illustrated in FIG. 2, fish transformed with a plasmid vector encoding IHNV G under transcriptional control of IRF1A (pIRF1A-G) demonstrated increased resistance to IHNV. Surprisingly, these fish demonstrated better resistance to IHNV infection than fish inoculated with a plasmid vector encoding IHNV G under transcriptional control of the cytomegalovirus promoter (pCMV-G). These results are further described in Example 3 below. Similar immune responses are produced when IRF1A is coupled to an antigenic epitope other than IHNV G, such as the antigens listed above.

While these polypeptides illustrate the invention, the invention should not limited to only IHNV G or IHNV M. Other suitable antigens for inducing an immune response in fish and other polypeptides capable of inducing cell death are described herein. Additionally, the sequence of the wild-type IHNV G or IHNV M polypeptide (i.e., a “native” IHNV G or IHNV M sequence) can be modified by conservative substitutions, such as substituting a hydrophobic amino-acid residue with another hydrophobic residue or substituting a hydrophilic residue with another hydrophilic residue.

Random peptide sequences of at least about 20 amino acid residues (or about 6 to about 8 amino acid residues for a hapten) that would be expected to exhibit immunogenicity can be identified by computer analysis, then screened for reactivity with fish antibody or mammalian antibody, including but not limited to, combinatorial chemistry and expression library approaches (e.g. phage expression libraries, including those in which a peptide is joined in reading frame to an outer structural protein of the phage). Exemplary assays, e.g., western blot analysis for reactivity and ELISA, that are useful for screening phage expression libraries for polypeptides that mimic IHNV G or IHNV M are disclosed, in the scientific literature (see, e.g., Xu, L., et al., J. Virol. 65(3):1611-15 (1991) and Lee J. Y., et al., J. Gen. Virol. 77(Pt. 8):1731-37 (1996)).

Suicide Vectors

Certain embodiments of the invention utilize a nucleic acid composition containing an expression control sequence operably linked to a nucleic acid sequence encoding a polypeptide capable of inducing programmed cell death (PCD). Exemplary polypeptides capable of inducing PCD in animal cells include matrix proteins of rhabdoviruses, such as the IHNV matrix protein (IHNV M); the IPNV VP-2 protein; Adenovirus 5 E1A protein; and Hepatits B virus X protein.

In particular embodiments, the nucleic acid sequence encoding a polypeptide capable of inducing PCD is a nucleic acid sequence encoding the IHNV M polypeptide, or a conservative variant thereof (see, e.g., Chiou, P. P., et al., J. Virol., 74(16):7619-27 (2000), herein incorporated by reference). In more particular embodiments, the expression control sequence operably linked to the nucleic acid sequence encoding IHNV M is an inducible promoter, such as a promoter selected from the group consisting of metallothionein promoters, heat shock promoters, carbonic anhydrase promoters, and haptoglobin nucleic acid promoters.

Exemplary promoters include the Oncorhynchus nerka metallothionein-B promoter (MT), the sequence of which is shown in GenBank via Accession No. X67307, herein incorporated by reference, and the fish heat shock protein 70 highly inducible promoter (HSP70). HSP70 sequences have been identified in rainbow trout (Kothary R K, et al., Mol. Cell. Biol., 4(9):1785-91 (1984), herein incorporated by reference), medaka (Arai A, et al., Jpn J Genet., 70(3): 423-33 (1995), herein incorporated by reference) and zebrafish (Lele Z, et al., Dev Genet. 21(2):123-33 (1997)). Robert Devlin, Dept. Fisheries and Oceans, West Vancouver (Canada) provided the rainbow trout MT promoter for construction of nucleic acid vectors described herein. The HSP70 promoter was obtained by cloning from rainbow trout.

In some embodiments, the nucleic acid composition contains an inducible promoter operably linked to an IHNV M sequence and a second expression control sequence operably linked to a second nucleic acid sequence. In specific embodiments, the second nucleic acid sequence encodes an antigenic epitope. In one specific, non-limiting example, the nucleic acid composition is be a plasmid containing the MT promoter operably linked to an IHNV M sequence and an IRF1A promoter operably linked to an IHNV G sequence.

Such a nucleic acid composition, commonly called a “suicide vector,” can be considered a nucleic acid that both stimulates an immune response in the host and elicits autodestruction after the the nucleic acid sequence(s) encoding one or more antigenic epitopes are expressed. In some embodiments, the suicide vector is maintained as an extrachromosomal element. Autodestruction can be induced by inducing the expression of the IHNV M sequence from the extrachromosomal element, which expresses IHNV M polypeptide and induces PCD. The nucleic acid composition within the cell is destroyed along with the cell and eliminated via the same physiological and biochemical processes as any cell that died naturally within the fish.

PCD is a physiological, energy-consuming mechanism leading to suicide of the cell. One subset of PCD is apoptosis, characterized by morphological changes of a dying cell that include plasma membrane blebbing, nuclear and cytoplasmic shrinkage, and chromatin condensation. Cells that undergo apoptosis often fragment into membrane-bound apoptotic bodies that are readily phagocytosed and digested by macrophages or by neighboring cells without generating an inflammatory response (see Zhu L and Chun J,. editors, Apoptosis detection and assay methods (Eaton Publishing Company/Bio Techniques Books Division, 1998)).

Cell death is accomplished by the activation of endonucleases that fragment the cell's nuclear DNA into internucleosomal fragments (see, e.g., Zhang, J. H., and Xu, M., Cell Res., 10(3):205-11 (2000)). Due to the generation of such fragments, the DNA of apoptotic cells typically migrates as a ladder of 180-200 bp multimers on an agarose gel. IHNV infection produces oligonucleosomal DNA fragmentation in cultured cells. During IHNV infection, the IHNV M protein inhibits host transcription and triggers PCD (see Chiou P. P., et al. (2000)).

Since such a nucleic acid has an autodestruction capability, the nucleic acid can be substantially eliminated from the fish at virtually any time after introduction into the fish. These suicide vectors allow stimulation of an immune response in fish—including, but not limited to, stimulation of a prophylactic immune response—while removing substantially all of the traces of the nucleic acid after transfection. therefore, a suicide vector can be introduced into fish, the subsquent immune response to the antigen expressed by the vector can occur, and then the vector can be introduced to autodestruct and be eliminated from the fish by the time the fish is readied for consumption.

Additionally, because these suicide vectors can be substantially eliminated from the fish after the immune response is induced, probabilities of tolerization, development of anti-DNA antibodies, or integration of plasmid DNA into the genome of the vaccinated fish or other organisms can be reduced. However, while extrachromosomal maintenance of these vectors can offer certain advantages, the invention is not limited to extrachromosomal maintenance of the suicide vectors. In some embodiments, the suicide vector integrates into the chromosomal DNA.

These vectors can be modified in a manner to direct their integration into the genome. For example, retroviral long-terminal repeat (LTR) sequences and a sequence encoding the corresponding retroviral integrase could be added to a suicide vector.

In addition to introducing nucleic/acid sequences encoding antigens, these nucleic acid compositions can be used for other purposes, such as administering growth hormone genes, lytic-peptide genes, cytokine genes, beta-ketolase genes, growth factor genes, genes encoding enzymes, and antimicrobial genes. Furthermore, if extrachromosomal vectors are used, these suicide vectors offer a simpler, more efficient alternative to the traditional, lengthy process of producing transgenic animals by introducing transgenes into the host animal's genomic DNA. Such suicide vectors demonstrate extremely low probabilities of being integrated into the host genomic DNA (see, e.g., Anderson, E., et al., (1996)), and, therefore, can be substantially eliminated from the host animal's tissues. This feature can help alleviate public concerns about genetically modified organisms used in food production. For example, one or more suicide vectors could be used to augment important traits in fish during the production phase, yet later eliminated prior to market.

The MT promoter is used in specific embodiments. This promoter is transcriptionally active in a wide variety of fish tissues, including muscle tissue, in a variety of fish species, including rainbow trout, coho salmon, chinook salmon, amago salmon, chum salmon, sockeye salmon, Atlantic salmon, arctic char, brown trout, cutthroat trout, brook trout, catfish, tilapia, sea bream, seabass, flounder, and sturgeon. MT has been shown to be inducible by metals, including zinc, in fish cells. Fish have been shown to ingest zinc added to the surrounding water in amounts sufficient to induce MT (see, e.g., Chan W K, and Devlin R H, Mol Mar Biol Biotechnol. 2(5):308-18 (1993); Inoue K, et al., Biochem Biophys Res Commun. 185(3):1108-14 (1992)). Therefore, fish transformed with a nucleic acid composition described herein carrying the MT promoter, such as the suicide vectors described above, are capable of ingesting amounts of zinc sufficient to induce the MT promoter.

Other embodiments utilize nucleic acid compositions having promoters other than MT, such as a heat shock promoter (HSP). For example, a nucleic acid composition containing the HSP70 promoter (described above) operably linked to a nucleic acid sequence encoding the IHNV M protein can be used to transform fish. In such an embodiment, application of heat—such as an increase in water temperature, application of a heated needle, or the use of a low-intensity laser—can induce expression of IHNV M, resulting in cell death and elimination of the nucleic acid composition from the muscle tissue of the transformed fish.

The suicide vectors are capable of reducing the number of cells in a cell culture, tissue, or organism. In some embodiments, cells in a cell culture are transformed by a nucleic acid composition including an inducible promoter operably linked to a nucleic acid sequence encoding the IHNV M polypeptide, or a conservative variant thereof. In other embodiments, the inducible promoter is generally coupled to a nucleic acid encoding a polypeptide, other than IHNV M, that is capable of inducing PCD. In other embodiments, the transformed cells are not in a cell culture, but are instead located in the tissues or organs of an animal, such as a fish. In any of these embodiments, the inducible promoter drives expression of the polypeptide that, in turn, leads to the death of the transformed cell, thereby reducing the number of cells in the animal or cell culture.

In embodiments where the cells are located in a fish, the fish can belong to a particular species, such as rainbow trout, coho salmon, chinook salmon, amago salmon, chum salmon, sockeye salmon, Atlantic salmon, arctic char, brown trout, cutthroat trout, brook trout, catfish, tilapia, sea bream, seabass, flounder, and sturgeon. Particular embodiments utilize rainbow trout.

Transgenic Fish

In certain embodiments of the invention, any of the nucleic acid compositions described above is used to transform fish tissue to produce a transgenic fish.

In some embodiments, a nucleated cell of the transgenic fish is transformed with a nucleic acid sequence at least 70% identical to SEQ ID NO:1 operably linked to a heterologous nucleic acid sequence wherein SEQ ID NO:1 functions as a promoter under appropriate conditions. If activated, the nucleic acid sequence at least 70% identical to SEQ ID NO:1 can drive the expression of transgenes encoded by the heterologous nucleic acid sequence. For example, in specific embodiments, the heterologous nucleic acid sequence encodes an antigenic epitope, such as IHNV G, and the fish produces an immune response to the antigenic epitope. In such embodiments, the fish exhibits an increased resistance to infection by IHNV as compared to a non-transformed fish of the same species. In other embodiments, the heterologous nucleic acid sequence encodes a transgene other than an antigen, such as a growth hormone, lytic-peptide, growth factor cytokine, or beta-ketolase. In any particular embodiment, the heterologous nucleic acid sequence can express a conservative variant of a polypeptide. As just one, non-limiting example, the nucleic acid sequence at least 70% identical to SEQ ID NO:1 can drive expression of a conservative variant of the IHNV G protein.

In some embodiments, the heterologous nucleic acid sequence contains multiple ORFs. In particular embodiments, these multiple ORFs are under the control of different expression control sequences. For example, a composition can include the nucleic acid sequence at least 70% identical to SEQ ID NO:1 operably linked to a nucleic acid sequence encoding IHNV G (or a conservative variant thereof) and a promoter, such as the MT promoter, operably linked to IHNV M (or a conservative variant thereof). Particular embodiments utilize transgenic fish transformed with the vectors described above. In one specific, non-limiting example, the transgenic fish is transformed with a vector substantially similar to the vectors illustrated in FIG. 4.

Certain embodiments utilize transgenic fish of particular species, such as rainbow trout, coho salmon, chinook salmon, amago salmon, chum salmon, sockeye salmon, Atlantic salmon, arctic char, brown trout, cutthroat trout, brook trout, catfish, tilapia, sea bream, seabass, flounder, and sturgeon. Particular embodiments utilize transgenic rainbow trout.

Eliciting an Immune Response in Fish

As described above, some embodiments utilize nucleic acid compositions containing nucleic acid sequences encoding antigenic epitopes. In such embodiments, the nucleic acid composition includes a sequence at least 70% identical to SEQ ID NO:1 that functions as a promoter. This promoter is operably linked to a second nucleic acid sequence encoding an antigenic epitope, thereby driving expression of the second nucleic acid sequence and eliciting an immune response to the antigenic epitope in the fish. In particular embodiments, the antigen expressed is IHNV G, which elicits an immune response against IHNV. In alternative embodiments, the antigen expressed is VCV-G (GenBank Accession No. U18101, herein incorporated by reference) which elicits and immune response against spring viremia of carp virus in carp. In alternative embodiments, the antigen expressed is encoded by all or a portion of the VP2 nucleic acid of IPNV (GenBank Accession No. S58170, herein incorporated by reference), which elicits an immune response against IPNV. In other alternative embodiments, the antigen expressed is the VHSV G protein GenBank Accession No. X66134, herein incorporated by reference) which elicits an immune response against VHSV. In still other alternative embodiments, the antigen expressed is the Infectious Salmon Anemia Virus (ISAV) hemagglutinin or neuraminidase protein, which elicits an immune response against ISAV. In yet other alternative embodiments, the antigen expressed is the p57 nucleic acid of Renibacterium salmoninarum (GenBank Accession No. Af123890, herein incorporated by reference), which elicits an immune response against Renibacterium salmoninarum. In yet other alternative embodiments, the antigen expressed is the flagellin A nucleic acid of Vibrio antuillarum (GenBank Accession No. L47122, herein incorporated by reference), which elicits an immune response against V. anguillarum. In still other alternative embodiments, the antigen expressed is the flagellin protein of Aeromonas salmonicida (GenBank Accession No. AF002709, herein incorporated by reference), or the prepilin TaA and type IV pilus assembly proteins of Aeromonas salmonicida (GenBank Accession No. AF059248, herein incorporated by reference), which elicit immune responses against A. salmonicida.

In some embodiments, the nucleic acid composition includes sequences other than the sequence at least 70% identical to SEQ ID NO:1 and the sequence encoding an antigenic epitope. In particular embodiments, the composition includes additional expression control sequence operably linked to a third nucleic acid sequence, wherein the third nucleic encodes a polypeptide. The additional expression control sequence can be a promoter, and the third nucleic acid sequence can encode a protein capable of initiating cell death, a growth hormone, a lytic-peptide, acytokine, a beta-ketolase, a reporter substance, or any other peptide of interest. In more specific embodiments, the expression control sequence is an inducible promoter, a metallothionein promoter, heat shock promoter, carbonic anhydrase promoter, or haptoglobin nucleic acid promoter, and the third nucleic acid sequence encodes the IHNV M protein, or a conservative variant thereof.

In any such embodiment, the fish utilized can belong to a particular species, such as rainbow trout, coho salmon, chinook salmon, amago salmon, chum salmon, sockeye salmon, Atlantic salmon, arctic char, brown trout, cutthroat trout, brook trout, catfish, tilapia, sea bream, seabass, flounder, and sturgeon. Particular embodiments utilize rainbow trout.

### EXAMPLES

The following examples are provided to illustrate particular features of certain embodiments. The scope of the invention should not be limited to those features exemplified.

### Example 1

### Maintaining IHNV

IHNV itself can be maintained in tissue culture from the Chinook salmon embryonic cell line (CHSE-214). Engelking H M, and Leong J C, Virology, 109(1):47-58 (1981). The IHNV isolate disclosed herein was obtained in 1983 from juvenile rainbow trout at the Rangen Research Laboratories, Idaho, USA. Hsu Y L, et al., Appl Environ Microbiol. 52(6):1353-61 (1986). The virus was propagated in CHSE-214 cell monolayers at a multiplicity of infection of 0.001 at 15° C. The medium was harvested when viral cytopathic effects become apparent and cellular debris was removed by low-speed centrifugation. Quantification of clarified virus was performed by tissue culture infectious dose₅₀assay (TCID ₅₀/ml) or by plaque asssay.

IHNV is one source for IHNV G and IHNV M nucleic acids or polypeptides. However, these nucleic acids and polypeptides also can be produced by other means, such as chemical synthesis or recombinant expression.

### Example 2

### Determination of the IRF1A Sequence

A cDNA library was made to mRNA from rainbow trout gonad cells (RTG-2) after interferon induction with dsRNA (Trobridge G D, and Leong J A, J Interferon Cytokine Res. 15(8):691-702 (1995); Mourich D V, et al., Immunogenetics 42(5):438-9 (1995)). This library yielded the sequence of the trout interferon regulatory factor 1 (IRF1A) as presented in FIG. 1 and SEQ ID NO:1. This clone was used to screen genomic libraries for promoter sequences using “Genome Walker” technology (Clontech, Palo Alto, Calif.).

Pure genomic DNA was isolated from RTG-2 cells induced with poly I.C. using proteinase K and phenol/chloroform extraction and subsequently digested with four different restriction enzymes (EcoRV, DraI, PvuII, SspI) that recognize a 6-base site leaving blunt ends. Following digestion, each pool of DNA fragments was ligated to GenomeWalker adaptors. The genome fragments containing the trout promoters were used as templates to PCR amplify a genomic sequence with a rightward primer specific for the selected cDNA and the leftward primer to the adaptor sequence. This Genome Walker library was used as template to amplify by PCR the promoter regions upstream of the rainbow trout IRF1A nucleic acid. Genome Walker products were directly cloned into a TA-cloning vector, pCR2.1 (Invitrogen, Palo Alto, Calif.) and subsequently sequenced. Nucleotide sequence and elements of the rainbow trout IRF1A promoter are shown in FIG. 1.

### Example 3

### Effectiveness of IRF1A-IHNV G Construct

A vector containing the IHNV-G nucleic acid linked to the trout IRF1A promoter was constructed, injected intramuscularly into rainbow trout fry, and evaluated for its efficacy in inducing an immune response to IHNV. Several experimental groups were compared. Rainbow trout (0.4 g) were vaccinated with 5 μg of purified plasmid DNA in a volume of 10 μl as described by Anderson et al. (1996), Supra. At 30 days post-vaccination, triplicate sets of 60 fish (0.7 g) from each treatment were challenged by static immersion with 10⁵ pfu/ml of the IHNV-RA strain described in Hsu, Y. L., et al., Supra. Mortalities were recorded for 30 days and are shown in FIG. 2. The IRF1A trout promoter was found to induce a better immune response to IHNV than other promoters assayed. About 80.5% of the animals receiving the IRF1A-G survived an IHNV challenge.

FIG. 2 illustrates cumulative mortalities of fish injected with plasmid DNA containing the IRF1A promoter and the IHNV G nucleic acid and subsequently challenged with IHNV. PBS refers to the control group. pMx-G refers to fish injected with a plasmid containing the Mx promoter driving the expression of the IHNV glycoprotein nucleic acid. pMxSpe-G refers to a plasmid with the Mx promoter that has been altered in its interferon sensitive response element which has been turned into a SpeI restriction site. pcDNA3 is a standard plasmid available for purchase from Invitrogen (Carlsbad, Calif.). This plasmid, which is the negative control, contains the CMVIEP and a multiple cloning site for insertion of a nucleic acid of interest. The expression of the nucleic acid of interest is driven by the CMVIE promoter. pIRF1A-G refers to fish injected with plasmid DNA containing the IRF1A promoter operably linked to the IHNV G nucleic acid. pIRF1A-G refers to fish injected with plasmid DNA containing the CMVIE promoter operably linked to the IHNV G nucleic acid.

Rainbow trout at an average weight of 0.7 g were exposed to 10⁵ pfu/ml IHNV 30 days after immunization with PBS, pcDNA3, pCMV-G, pMx-G, pMxSpeI-G and pIRF1A-G. Additional fish from each treatment group were mock challenged with PBS, although no significant mortality occurred (data not shown).

### Example 4

### Persistence of IHNV G Expression

A plasmid containing the CMV promoter driving IHNV G expression remained in the muscle tissue of rainbow trout for at least 120 days after injection.

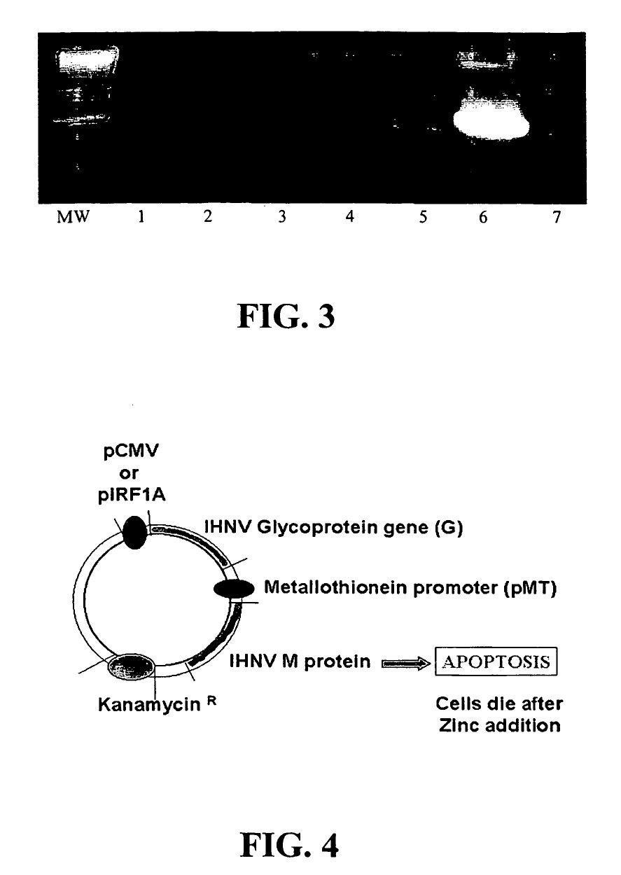

FIG. 3 illustrates the results of PCR analysis used to detect the IHNV glycoprotein nucleic acid in 5 fish injected with pCMV-G at 120 days after injection. Total DNA was extracted from 25 mg of frozen muscle tissue using the QIAamp DNA Mini Kit (Qiagen Ltd, Crawley, UK) according to the manufacturer's instructions. The 1589 bp fragment was present in all five fish (numbers 1, 2, 3, 4 and 5), but not in the fish injected with PBS (control fish, data not shown). As a PCR positive control, we show the PCR results of the plasmid originally used to inject fish, pCMV-G at number 6. Number 7 is the PCR negative control.

Specific primers (5′-AACGCAACTCGCAGAGACC-3′, SEQ ID NO: 2, and 5′-GTCTGGTGGG GAGGAAGTGAA-3′, SEQ ID NO: 3) were used to amplify by PCR a fragment of the G nucleic acid from 100 ng of total DNA using the following conditions: 94° C. 30 seg, 55° C. 1 min and 72° C. 2 min. The PCR products were run on 0.8% agarose gels and photographed with Polaroid film. These photographs were later processed on a digital scanner to produce digital images. The amplified products were sequenced and digested with restriction enzymes to verify that they correspond to the IHNV G nucleic acid.

### Example 5

### Construction of Expression Vectors Containing IHNV M

Two “suicide vectors” were constructed: (1) pCMV-G-pMT-M expresses the IHNV-G nucleic acid from the cytomegolovirus immediate early promoter and IHNV-M from the rainbow trout metallothionein promoter (pMT); and (2) The vector, pIRF1A-G-pMT-M, was designed to express the IHNV-G from the rainbow trout IRF1A promoter and IHNV-M from the rainbow trout metallothionein promoter.

### Example 6

### Transient Transfection Assays

In vitro experiments in epithelioma papulosum cyprini cells (EPC) were performed to verify the mechanisin of action of the “suicide vectors”.

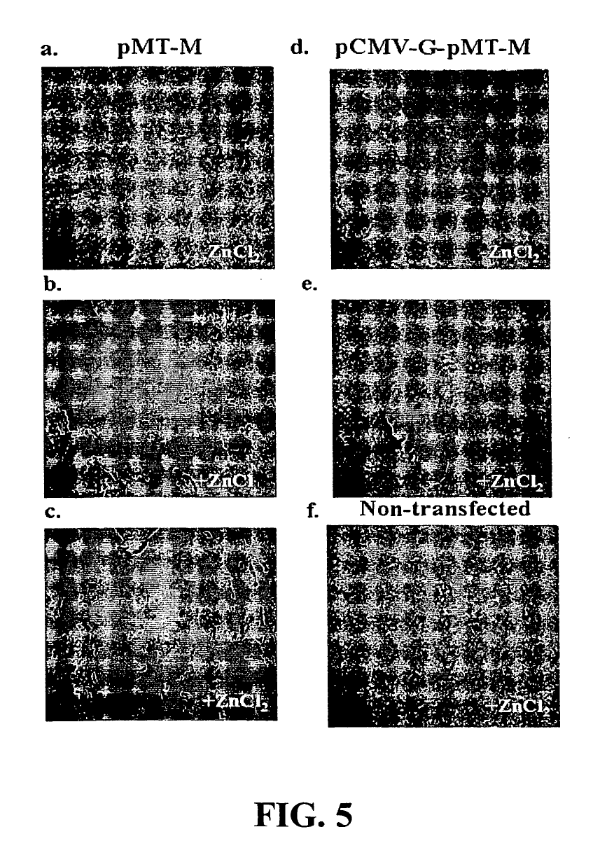

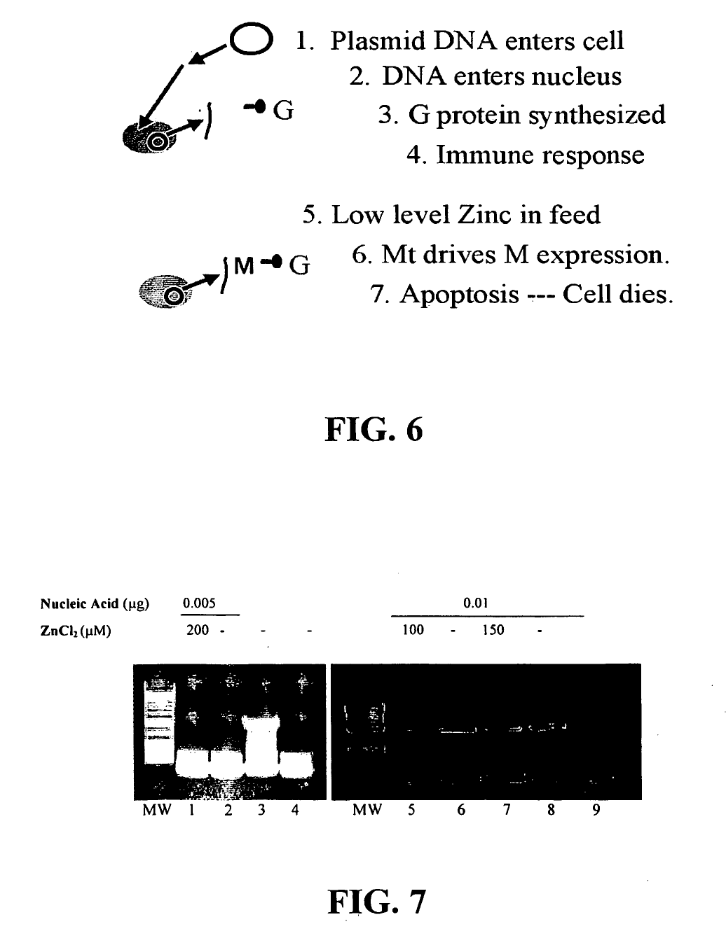

EPC cells were transfected with pCMV-G-pMT-M or pMT-M, a plasmid containing the IHNV M nucleic acid under the metallothionein promoter. Cell transfection assays were made using Lipofectamine Plus following the instructions of the manufacturer (Gibco, BRL, Gaithersburg, Md.). After 48 hours, the cells were treated with 100 μM ZnCl₂, an inducer of the MT promoter. Results of the transfection assays are shown in FIG. 5.

FIG. 5 illustrates a transient transfection assay of in epithelioma papulosum cyprini (EPC) cells with two particular suicide vectors, pMT-M (a,b,c) and pCMV-G-pMT-M (d,e) with and without ZnCl₂. (100 μM). Cells transfected with the different constructs were left uninduced (a,d) or were treated with 100 μM ml of ZnCl₂ at 48 h. post-transfection (b,c,e,f).

IHNV M protein was expressed in cells transfected with pCMV-G-pMT-M inducing programmed cell death. The transfected cells could be seen shrinking, rounding up, and subsequently detaching from the plate as a consequence of cell death. As positive control, EPC cells were transfected with pMT-M and the effect of ZnCl₂ addition was even more drastic. A marked reduction in the viable cell number by the addition of ZnCl₂ was observed. Considerable cell death had occurred, as reflected in a large number of rounded up or detached cells. Untransfected EPC control cells were not obviously affected after addition of ZnCl₂.

### Example 7

### Time Course of G and M Proteins Expression in Transfected Cells

The capacity of the plasmids to express M and G IHNV proteins is confirmed in transfected EPC cells at different times after ZnCl₂ addition by a Western-blot analysis using an anti-IHNV polyclonal antibody. EPC cells are transfected with 1 μg each of pCMV-G-pMT-M, pIRF1A-G-pMT-M, pMT-M and pCMV-G using lipofectamine Plus (Gibco BRL, Gaithersburg, Md.) according to the manufacturer's instructions. Approximately 48 hours after transfection, cells are treated with 100 μM ZnCl₂ or left untreated. A time course study for G and M IHNV proteins in EPC cells maintained in 100 μM ZnCl₂ will be performed. After 0, 2, 6, 8, 12, 24, 48 and 72 hours post-ZnCl₂ addition, cells are collected and the protein concentration determined by a Bradford protein assay (Biorad). Samples for Western blot analysis are prepared as described in Kim, et al., J. Virology 74(15):7048-54 (2000).

### Example 8

### G and M mRNA Detection in Transfected Cells/Northern Blot Analysis

mRNA of the G and M proteins is detected in transfected cells. Total RNA is prepared from cells transfected with pCMV-G-pMT-M, pIRF1A-G-pMT-M, pMT-M and pCMV-G and treated with 100 μM ZnCl for (0, 2, 6, 8, 12, 24 ,48 and 72 hours) using the Trizol reagent as recommended by the manufacturer (Life Technologies). Polyadenylated RNA is separated from total RNA with PolyAtract mRNA Isolation system (Stratagene). For Northern-blot analysis 4, μg of the RNA samples are electrophoresed on a 2% formaldehyde-denaturing agarose gel and then transferred in 20×SSC overnight to nitrocellulose membranes by standard blotting techniques (Sambrook et al.,2001). The G and M IHNV transcripts are detected with 1589 bp G- and 140 bp M-specific dsDNA probes obtained by PCR, and which are one-step labeled with horseradish peroxidase (HRP) using a North2South direct HRP system (Pierce). The same kit is used for hybridization and chemiluminescent detection. The hybridized filters are washed under stringent conditions and exposed to X ray film (Kodak). The relative intensity of the specific signals are obtained by scanning the blot in a PhosphorImager scanner (Molecular Dynamics). A reduction in the expression of the G and M genes is observed over time as consequence of the IHNV M induction of cell death.

### Example 9

### Induction and Evaluation of Apoptosis

The capacity of cells transfected with pMT-M, pCMV-G-pMT-M and pIRF1A-G-pMT-M to undergo apoptosis by treatment with 100 μM ZnCl₂ for different time points (1, 2, 6, 8, 12, 24 and 48 hours) is evaluated. Untransfected control cells are subjected to the same treatment. Evaluation of apoptosis is performed by using the following methods.

TdT incorporation of labeled nucleotides into DNA strand breaks (TUNEL assay) (Apoptosis Detection System, Promega). This system is a fluorescent TUNEL assay that measures apoptotic DNA fragmentation by directly incorporating fluorescein-12-dUTP at the 3′-OH DNA ends using Terminal Deoxynucleotidyl Transferase (TdT), which forms a polymeric tail. The fluorescein-dUTP-labeled DNAs from tranfected cells are then visualized directly by fluorescence microscope or quantitated by flow cytometry. Samples are analyzed by confocal microscopy (Leica TCS4D) and by a FACScan Flow cytometer (Becton Dickinson).

DNA fragmentation assay. In contrast to the formation of DNA ladders found in apoptotic cells, necrotic cells show a smear of low-molecular-weight DNA staining due to the random cleavage of DNA by nonspecific DNAses. DNA is extracted from transfected cells as described by Takizawa et al., J. Gen. Virol., 74(pt. 11):2347-55 (1993). Medium and cells can harvested from dishes and centrifuged. The pelleted cells are resuspended in cold lysis buffer (Promega) and incubated on ice for 30 min. After centrifugation, the supernatant is extracted with an equal volume of buffered phenol. DNA is then precipitated with ethanol and treated with 1 mg/ml RNase A (Sigma) for 30 min at 37° C. Finally, aliquots of the DNA samples are electrophoresed though a 2% agarose gel and stained with ethidium bromide.

Transfection frequencies ranging from 0.5 to 2% are routinely observed, with an occasional transfection rate of 10% in CHSE-214 and EPC cells.

### Example 10

### Toxicity of ZnCl₂

The elimination of the cells containing suicide vectors after addition of low concentrations of ZnCl₂ to the water was tested by polymerase chain reaction (PCR) analysis.

Three different concentrations of ZnCl₂ (1 μM, 10 μM, 100 μM) were assayed and analyzed for their toxic effects in rainbow trout fry (0.8 g). Forty-eight hours after the addition of ZnCl₂ to the water, all fish immersed in 100 μM ZnCl₂ had died, but no mortalities were recorded in the other treatments.

### Example 11

### Elimination of Vector DNA from Fish After Induction of Apoptosis

Rainbow trout fry (0.8 g) were injected intramuscularly with 10 ng of the suicide vector (pCMV-G-pM-M1). After 48 hours, ZnCl₂ (10 μM) was added to the aquarium water. After another 48 hours, DNA was isolated from the injection place. PCR analysis with G IHNV specific primers (SEQ ID NO: 2 and SEQ ID NO: 3) were performed to verify that the vector was eliminated from muscle tissue of the injected fish. The suicide vector could not be detected in fish that had been treated with ZnCl₂, but the vector persisted in all untreated fish assayed in the same manner.

### Example 12

### Elimination of Vector DNA from Fish After Induction of Apoptosis

The same experiment described in Example 11 is repeated with different concentrations of suicide vector (4 μg , 1 μg, 0.1 μg, 0.01 μg and 0.001 μg). DNA is extracted and PCR assay conducted in the same manner.

### Example 13

### Specific Expression Vectors

FIG. 4 illustrates two different expression vectors. The first is the plasmid pCMV-G-pMT-M. This plasmid contains a nucleic acid sequence encoding IHNV G under the control of the CMV promoter and a nucleic acid sequence encoding IHNV M under the control of Oncorhynchus mykiss MT. The MT promoter was inserted in the NotI restriction site of pCMV-G expression vector. The second composition illustrated in FIG. 4 is similar to the first, except that the IRF1A promoter is used in place of the CMV promoter.

### Example 14

### Zinc Induces the MT Promoter

The activity of the rainbow trout MT promoter was examined by microinjecting plasmids containing pMT operably linked to the CAT reporter into fertilized eggs of medaka, as described in Inoue, et al., Supra. CAT activities were determined in hatchlings with and without stimulation with zinc. Only small trace levels of CAT activity were detected in unstimulated fish, but CAT activity increased in fish stimulated with zinc. Additionally, we have found that a 100 μM zinc dose induces nucleic acid expression mediated by the metallothionein promoter in fish cells without toxicity.

### Example 15

### Heat Induces the HSP70 Promoter

Application of heat induces the HSP70 promoter. Heat shock for 1 hour at 37° C. resulted in a 16-fold stimulation of β-galactosidase expression driven by the tilapia HSP70 promoter in EPC cells, while no basal level of expression was evident in the uninduced cells (Molina et al., FEBS Lett. 474(1):5-10 (2000). Nucleic acid expression was also induced by focusing a sublethal laser microbeam onto specific cells in zebrafish embryos (Halloran et al., Development, 127(9):1953-60 (2000). Not only were the targeted cells undamaged, but they appeared to develop normally.

### Example 16

### Vaccination Trials

To assess the protective effects of the various “suicide vectors” against IHNV, in vivo lethal challenge assays are conducted on rainbow trout fry (Oncorhynchus mykiss) provided by the Food Toxicology and Nutrition Laboratory at Oregon State University. To acclimate the animals prior to undergoing experimental procedures, the fish are held at the Oregon State University Salmon Disease Laboratory in tanks supplied with 12° C. specific pathogen free well water flowing at a rate of 0.5 L/min and fed daily ad libitum with trout pellets.

### Example 17

### Vaccination Protocol

Several experimental groups of fish are studied in these laboratory trials, including: 1) control, unvaccinated fish, 2) control phosphate buffered saline (PBS) injected fish, 3) control fish injected with pcDNA3, 4) fish injected with pCMV-G-pMT-M, 5) fish injected with pIRF1A-G-pMT-M and 6) fish injected with pMT-M. Rainbow trout (mean weight, 0.4 g) are anesthetized by immersion in a 100 μg/ml of tricaine methane sulfonate (MS-222, Finquel) and injected intramuscularly at the base of the dorsal fin with 10 μl of phosphate buffered saline (PBS) containing 5 μg of purified plasmid DNA as previously described (Anderson et al.,1996, Supra). Each preparation is administered to a group of 350 individuals placed in separate 100 L aquaria. Great care is taken to ensure that the fish are all injected identically. To eliminate the possible variability due to DNA preparation, each assay is performed with the same preparation of plasmid DNA. Tank locations are randomized to avoid possible effects of tank position.

### Example 18

### Challenge Assays

The protective immunity elicited by a suicide vector is evaluated through survival of immunized fry after challenge with live virus. At 30 days post-vaccination (dpv), triplicate sets of 60 fish from each treatment are challenged by static immersion with 10⁵ pfu/ml of the IHNV-RA strain for 5 hours. Negative control fish, one tank of 60 fish for each treatment group, receives PBS instead of infectious virus. Dead fish are collected daily and examined for visible signs of IHN disease. At least 10% of dead fish from each treatment are processed by standard methods to determine whether the fish died from an IHNV infection. The relative percent survival (RPS) for each group is calculated by the following formula: RPS=(1-[% mortality of vaccinated fish]/[% mortality of control fish])×100. A significant level of protection is seen in groups receiving pCMV-G-pMT-M, pIRF1A-G-pMT-M and pCMV-G when compared to the PBS, uninjected and pMT-M injected fish.

### Example 19

### Activation of the Metallothionein Promoter by ZnCl₂ Treatment

Different concentrations of ZnCl₂(10 mM, 5 mM, 2.5 mM, 1 mM, 100 μM, 10 M and 1 μM) were assayed and observed for their toxic effect in rainbow trout. Concentrations of ZnCl₂ higher than 10 μM were toxic for rainbow trout fry (1 g). After 30 days post-challenge, the correct dose (10 μM ZnCl₂) is added to 90 fish per treatment. After 48 hours, PCR analysis with IHNV G and M specific primers is performed to verify that the DNA vaccines have been totally eliminated from muscle tissue of the vaccinated and Zinc exposed fish.

### Example 20

### PCR Tracing

PCR amplification is a sensitive and specific method for the detection of expression vectors in fish tissues. Small quantities of plasmid DNA can be detect in the muscle tissue of a vaccinated fish using this method FIG. 3. Groups of fish injected with 5 μg of pCMV-G-pMT-M, pIRF1A-G-pMT-M, and pMT-M that received ZnCl₂ do not retain the suicide vector. Contamination is monitored by three negative controls assayed by PCR: water sample with no DNA, genomic DNA from animals PBS injected, and genomic DNA from animals injected with a plasmid, which amplification is not possible with the primers used. A control experiment is performed to determine the sensitivity of the PCR assay. Different concentrations of pCMV-G-pMT-M are injected in fish (10, 1 and 0.1 μg), and DNA is extracted and PCR assay conducted.

### Example 21

### Suicide Vector Construction

A suicide vector was constructed consisting of two operons: (1) an inducible fish promoter (pIRF1A) driving the expression of the viral glycoprotein (G) nucleic acid that induces protection; and (2) an inducible fish promoter (pMT) driving the expression of a nucleic acid that induces apoptosis (M). To construct the suicide vector, a cassette containing the IHNV matrix nucleic acid (M) under the control of the Oncorhynchus mykiss metallothionein promoter B (pMT) was inserted in the NotI restriction site of the pIRF1A-G expression vector. The new construction was confirmed by restriction enzyme and DNA sequence analysis.

### Example 22

### Ability of a Suicide Vector to Induce Apoptosis in Transfected Cells After ZnCl₂ Addition

Persistence of a DNA suicide vector (constructed according to Example 21) in transfected cells was assessed by a reverse transcriptase reaction coupled to PCR (RT-PCR). EPC cells were transfected with 0.01 μg or 0.005 μg of pIRF1A-G-pMT-M using lipofectamine Plus (Gibco BRL). Approximately 24 hours after transfection, the cells were treated with non-toxic concentrations of ZnCl₂ (100, 150, or 200 μM). Control cells were left untreated.

At 48 hours after the ZnCl₂ addition, total RNA was isolated and an RT-PCR assay using IHNV-G specific primers (5′-AACGCAACTCGCAGAGACC-3′ provided by SEQ. ID. NO: 2 and 5′-GTCTGGTGGG GAGGAAGTGAA-3′ provided by SEQ. ID. NO: 3) was performed. PCR conditions were: 94° C. 30 sec, 55° C. 1 min and 72° C. 2 min. The PCR products were run on a 0.8% agarose gels and photographed with Polaroid film (and later digitized using a scanner), as shown in FIG. 7.

Cells transfected with 0.01 μg of the suicide vector did not appear to be affected by the addition of ZnCl₂, regardless of the concentrations used. The 1589 bp fragment was obtained with (FIG. 7, lanes 5 and 7) and without (FIG. 7, lanes 6 and 8) the ZnCl₂ presence in the growth medium. However, when the cells were transfected with the minimum dose of nucleic acid assessed (0.005 μg) and treated with 200 μM of ZnCl₂ concentration (FIG. 7, lane 1), no PCR positive results were obtained. These results indicate a remarkable reduction in the expression of the G nucleic acid as consequence of the IHNV M induction of cell death.

### Example 23

### Persistence of Nucleic Acid

Transfection with low concentrations of the suicide vector is desirable to reduce the quantity of DNA that would persist in the transfected fish. To assess the minimum dose of suicide vector detectable by PCR, rainbow trout fry (about 0.3 g) were injected with 4.0 μg, 1.0 μg, 0.1 μg and 0.01 μg of the suicide vector. The transfected fish were kept in 25 L tanks supplied with 13° C. of non-chlorinated pathogen free water for two days after the injection. Total DNA was extracted from 25 mg of the muscle tissue using the QIAamp DNA Mini Kit (Qiagen), according to the manufacturer's instruction. Specific primers were used to amplify by PCR a fragment of the G nucleic acid from 100 ng of the total DNA using the following conditions: 94° C. 30 sec, 55° C. 1 min and 72° C. 2 min.

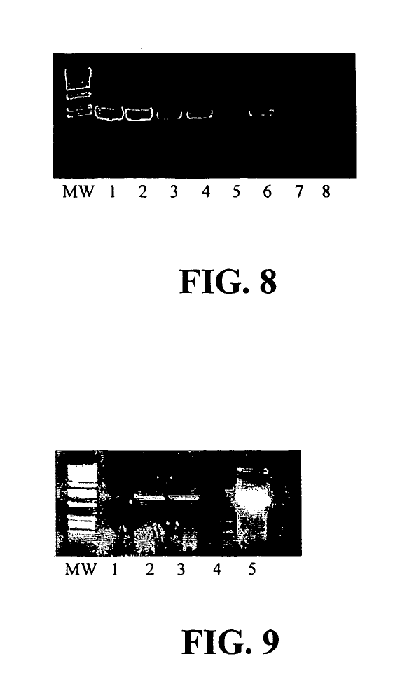

Using PCR, episomal (non-integrated) suicide plasmid DNA was detected in the muscle tissue of fish vaccinated with 4 μg, 1 μg and 0.1 μg, as shown in FIG. 8. No plasmid DNA was detected in fish injected with a treatment dose of 0.01 μg (FIG. 8, lanes 7 and 8).

### Example 24

### Persistence of Nucleic Acid in Fish After ZnCl₂ Treatment

The suicide vector has been tested in vivo to determine whether induction of programmed cell death also occurs in fish. PCR analysis with IHNV G primers was performed on fish at 48 hours post-exposure to ZnCl₂ to determine if apoptotic induction results in the loss of plasmid DNA.

As shown in FIG. 9, the suicide vector persisted in control fish (lanes 1 and 2), whereas the plasmid DNA was eliminated from muscle tissue of just one of the vaccinated and ZnCl₂ exposed fish (lanes 3 and 4).

Having illustrated and described the principals of the invention by several embodiments, it should be apparent that those embodiments can be modified in arrangement and detail without departing from the principals of the invention. Thus, the invention as claimed includes all such embodiments and variations thereof, and their equivalence, as come within the true spirit and scope of the claims stated below.

We claim:

1. A nucleic acid molecule comprising a nucleic acid sequence at least 70% identical to SEQ ID NO:1.

2. The nucleic acid molecule according to claim 1, wherein the nucleic acid sequence is at least 80% identical to SEQ ID NO:1.

3. The nucleic acid molecule according to claim 2, wherein the nucleic acid sequence is at least 90% identical to SEQ ID NO:1.

4. The nucleic acid molecule according to claim 3, wherein the nucleic acid sequence is at least 95% identical to SEQ ID NO:1.

5. The nucleic acid molecule according to claim 1, operably linked to a heterologous nucleic acid.

6. The nucleic acid moleucle of claim 1, wherein the heterologous nucleic acid encodes an antigenic epitope.

7. The nucleic acid molecule according to claim 5, wherein the heterologous nucleic acid encodes a infectious hematopoietic necrosis virus G polypeptide.

8. A vector comprising the nucleic acid molecule of claim 5.

9. The vector of claim 8, wherein the vector is a plasmid vector or a viral vector.

10. The vector of claim 8, further comprising an expression control sequence operably linked to a nucleic acid sequence encoding a polypeptide that induces programmed cell death.

11. The vector of claim 10, wherein the polypeptide that induces programmed cell death comprises an infectious hematopoietic necrosis virus M polypeptide.

12. The nucleic acid according to claim 10, wherein the expression control sequence comprises a promoter.

13. The nucleic acid according to claim 12, wherein the promoter is an inducible promoter.

14. The nucleic acid according to claim 12, wherein the promoter is selected from the group consisting of metallothionein promoters, heat shock promoters, carbonic anhydrase promoters, and haptoglobin nucleic acid promoters.

15. The nucleic acid according to claim 14, wherein the promoter is a metallothione promoter or a heat shock protein 70 (HSP70).

16. A host cell, comprising the nucleic acid of claim 5.

17. The host cell according to claim 16, wherein the cell is a fish cell.

18. The host cell according to claim 17, wherein the fish cell is selected from the group consisting of rainbow trout cells, coho salmon cells, chinook salmon cells, amago salmon cells, chum salmon cells, sockeye salmon cells, Atlantic salmon cells, arctic char cells, brown trout cells, cutthroat trout cells, brook trout cells, catfish cells, tilapia cells, sea bream cells, seabass cells, flounder cells, and sturgeon cells.

19. A transgenic fish, a nucleated cell of which comprises a nucleic acid sequence at least 70% identical to SEQ ID NO:1 operably linked to a heterologous nucleic acid sequence encoding an antigenic epitope, wherein the nucleic acid sequence at least 70% identical to SEQ ID NO:1 drives the expression of the antigenic epitope, and wherein the fish produces an immune response to the antigenic epitope.

20. The transgenic fish according to claim 19, wherein the fish is selected from the group consisting of rainbow trout, coho salmon, chinook salmon, amago salmon, chum salmon, sockeye salmon, Atlantic salmon, arctic char, brown trout, cutthroat trout, brook trout, catfish, tilapia, sea bream, seabass, flounder, and sturgeon.

21. The transgenic fish according to claim 19, wherein the antigenic epitope is an antigenic epitope of a infectious hematopoietic necrosis virus G protein or a conservative variant thereof.

22. The transgenic fish according to claim 21, wherein the fish exhibits an increased resistence to infection by infectious hematopoietic necrosis virus as compared to a non-transformed fish of the same species.

23. The transgenic fish according to claim 19, wherein the nucleated cell further comprises an expression control sequence operably linked to a nucleic acid sequence encoding a polypeptide that induces programmed cell death.

24. The transgenic fish of claim 23, wherein the polypeptide that induces programmed cell death is an infectious hematopoietic necrosis virus M protein or a conservative variant thereof.

25. The transgenic fish according to claim 23, wherein the expression control sequence comprises a promoter.

26. The transgenic fish according to claim 25, wherein the promoter is an inducible promoter.

27. The transgenic fish according to claim 26, wherein the promoter is selected from the group consisting of metallothionein promoters, heat shock promoters, carbonic anhydrase promoters, and haptoglobin nucleic acid promoters.

28. The transgenic fish according to claim 26 wherein the promoter is a metallothione promoter or a heat shock protein 70 promoter.

29. The transgenic fish according to claim 35 wherein the expression control sequence is operably linked to a heterologous nucleic acid sequence encoding a polypeptide.

30. A method of eliciting an immune response against an antigenic epitope in a fish, comprising
introducing into the fish a vector comprising the nucleic acid of claim 1, wherein said nucleic acid functions as a promoter, operably linked to a nucleic acid encoding an antigenic epitope,
thereby eliciting an immune response against the antigentic epitope in the fish.

31. The method according to claim 30, wherein the fish is a rainbow trout, a coho salmon, a chinook salmon, an amago salmon, a chum salmon, a sockeye salmon; an Atlantic salmon, an arctic char, a brown trout, a cutthroat trout, a brook trout, a catfish, a tilapia, a sea bream, a seabass, a flounder, or a sturgeon.

32. The method according to claim 30, wherein the antigenic epitope is an infectious hematopoietic necrosis virus G polypeptide or a conservative variant thereof.

33. The method according to claim 30, wherein the vector further comprises a nucleic acid sequence encoding a polypeptide that induces programmed cell death.

34. The method according to claim 33, wherein the polypeptide that induces programmed cell death is an infectious hematopoietic necrosis virus M polypeptide or a conservative variant thereof.

35. The method according to claim 33, wherein the nucleic acid sequence encoding a polypeptide that induces programmed cell death is operably linked to a promoter.

36. The method according to claim 35, wherein the promoter is an inducible promoter.

37. The method according to claim 36, wherein the promoter is selected from the group consisting of metallothionein promoters, heat shock promoters, carbonic anhydrase promoters, and haptoglobin nucleic acid promoters.

38. The method according to claim 37, wherein the promoter is a metallothionen promoter or a heat shock protein 70 promoter.

39. A method of producing a transgenic fish, comprising contacted a nucleated cell of the fish with an amount of the nucleic acid of claim 5 sufficient to introduce the nucleic acid into the cell, thereby producing the transgenic fish.

40. The method according to claim 39, wherein the fish is a rainbow trout, a coho salmon, a chinook salmon, an amago salmon, a chum salmon, a sockeye salmon, an Atlantic salmon, an arctic char, a brown trout, a cutthroat trout, a brook trout, a catfish, a tilapia, a sea bream, a seabass, a flounder, and a sturgeon.

41. A method of reducing the number of cells in a fish, comprising:
introducing into a fish a composition comprising an inducible promoter operably linked to a nucleic acid sequence encoding the a polypeptide that induces programmed cell death; and
inducing expression of the infectious hematopoietic necrosis virus M polypeptide from the inducible promoter,
thereby reducing the number of cells in the fish.

42. The method of claim 41, wherein the polypeptide that induces programmed cell death is an infectious hematopoietic necrosis virus M polypeptide or a conservative variant thereof.

43. The method according to claim 41, wherein the fish is a rainbow trout, coho salmon, a chinook salmon, an amago salmon, a chum salmon, a sockeye salmon, an Atlantic salmon, an arctic char, a brown trout, a cutthroat trout, a brook trout, a catfish, tilapia, a sea bream, a seabass, flounder, or a sturgeon.

44. The method according to claim 41, wherein the inducible promoter is selected from the group consisting of metallothionein promoters, heat shock promoters, carbonic anhydrase promoters, and haptoglobin nucleic acid promoters.

45. The method according to claim 44, wherein the promoter a metallothionine promoter or a heat shock protein 70 promoter.

46. The method according to claim 51 wherein the composition further comprises an expression control sequence operably linked to a nucleic acid sequence encoding a polypeptide.

47. The method according to claim 56 wherein the expression control sequences comprises the nucleic acid molecule of claim 1.

48. The method according to claim 58 wherein the polypepide is an antigenic epitope.

49. The method of claim 48, wherein the antigenic epitope is a infectious hematopoietic necrosis virus G polypeptide or a conservative variant thereof.


SEQUENCE LISTING (3 sequences)

SEQ ID NO: 1 - DNA, 1368 bp, Oncorhynchus mykiss
ctcgagcggccgccagtgtgatggatatctgcagaatcggcttactatagggcacgcgtggtcgacggcccgggctggtatcctttttttctattgggaaaaccctgggcggggggaacggagcaaaaagtccgcagcaggaaaataccctgcttttcctcattggactgagacacacccacacactgtattacacaactcccccgcgcgctccgagctcaaacacaacagtcattttatggaaaagacgggtgacgtgttcctgcttgacgaaggtttcctggcggattaacctctgcactgtaaatcagaacactgttaaatacgcgtttctgtatttatgcaatatgagacaattgagattaaagcagatcactccaagtccttataataaagtgcatggcagtttcatgctgttaacattcgaacgttggcgattcttgaatagtaatacataacatgattgcttaactttgtagcagttggcaattggtataagtgctttcttttgtaaacaagcagctcgtgtcagcagtgatttacacgaagtctgactggagtctgtggcaccctgtaccaactgaacaagtcaacacttcccctgtcggtgcttttaatacctacactatttccaggaagagcgcgacttctcagttcgattcggatttatttaaaagggcttgcctatgcctgggcttttgatgcttggcgggacataattgcaccaataatagaatagaaatacaacccagtggttgtatttggcatagttatgcaattaaataatggaatcgttttctctaatataattcaattaaattataggaatacgtgtggaaggattacttaactgataagatagcctgtttactatgcatcatctctatatattctataaacacaagttaaattaataaactatcaatagtcagcagaatttaacattttaactgaaaacaataagaaataaaaaaagcctagatattgtaaattatacttcgttatactgctacgatcaacatgcatagctactttattgggtctgttcacgccttatcatgactatgcatagttcgctcacaacggcatgatttctcggaaaccaggttgcctgtagtttgagcgcgccgtccttgttgtgacattcggcgcagatgcggactgctgccaaaaccgtatgcatgctactaagtggggaagtcccagcatctctgtataaaaaccattgttccttcagcagagtgtacacactgtcaatgaagaccagagtggatattacaaggaataacagaggaacaacttctcgctccatctttacagaagagaaagccgaattccagcacactggcggccgttactagtggatcc

SEQ ID NO: 2 - DNA, 19 bp, Artificial Sequence
  IHNV-G Specific PCR Primer
aacgcaactcgcagagacc

SEQ ID NO: 3 - DNA, 21 bp, Artificial Sequence
  IHNV-G Specific PCR Primer
gtctggtggggaggaagtgaa